(19) United States
(12) Patent Application Publication
(10) Pub. No.: US 2025/0349435 A1
(43) Pub. Date: Nov. 13, 2025

(54) SYSTEM, METHOD AND APPARATUS FOR ASSESSING EFFICACY OF NUTRACEUTICAL POLYPHENOLS UTILIZING AI

(71) Applicant: Todra Capital Inc., Vancouver (CA)

(72) Inventors: Sakwinder NARWAL, North Vancouver (CA); Miodrag ANDRIC, North Vancouver (CA); Felix AGAKOV, Edinburgh (GB)

(21) Appl. No.: 18/871,037

(22) PCT Filed: May 29, 2023
(86) PCT No.: PCT/CA2023/050736

§ 371 Date: Dec. 2, 2024

Related U.S. Application Data
(60) Provisional application No. 63/348,318, filed on Jun. 2, 2022.

(51) Int. Cl. G16H 50/30; G16H 10/60; G16H 20/60; G16H 40/67

(52) U.S. Cl. CPC G16H 50/30; G16H 10/60; G16H 20/60; G16H 40/67

(57) ABSTRACT

There is disclosed a system, method, and apparatus for assessing the efficacy of nutraceutical polyphenol supplements and dosage regimen utilizing artificial intelligence (AI). More particularly, in an embodiment, the present system, method and apparatus are configured to assess the efficacy of polyphenols derived from various different types of food sources. In an embodiment, user health data is collected from a plurality of sources including user wearable biosensors, user surveys input via mobile devices, optional user blood testing, and images of the user taken via the mobile devices.

### CROSS-REFERENCE TO RELATED APPLICATIONS

This application claims the benefit of U.S. Provisional Application Ser. No. 63/348,318 filed on Jun. 2, 2022.

### FIELD

The present invention relates to a system, method and apparatus for assessing efficacy of nutraceuticals, and more particularly polyphenols.

### BACKGROUND

Nutraceuticals are supplements which are derived from food sources containing bioactive components or ingredients which have nutritional or health benefits. Typically taken in a pill form or liquid form, nutraceuticals may be taken at regular dosing intervals in order to alleviate or prevent symptoms of chronic ailments, and may also improve overall life expectancy with better health.

One class of nutraceuticals is polyphenols which exhibit antioxidant properties, and which may potentially reduce the risk of development of various ailments by removing free radicals from the body. However, given the complex nature of nutraceutical polyphenols and the still incomplete understanding of exactly how they work, it may be difficult to assess the efficacy of taking nutraceutical polyphenol supplements, which may sometimes take months after a course of treatment has started before showing any noticeable effect. As a result, a regimen of beneficial nutraceutical polyphenol supplements may be stopped prematurely due to perceived lack of noticeable progress, and may be abandoned for other less effective options.

Therefore, what is needed is an improved system, method and apparatus for assessing the efficacy of nutraceutical polyphenol supplements which overcomes various limitations in the prior art.

### SUMMARY

The present disclosure relates to a system, method and apparatus for assessing the efficacy of nutraceutical polyphenol supplements and dosage regimen utilizing artificial intelligence (AI). More particularly, in an embodiment, the present system, method and apparatus assess the efficacy of nutraceutical polyphenol supplements derived from various different types of food sources, including specific polyphenol supplements which enhance bioavailability.

In an aspect, there is provided a system, method, and apparatus for collection of user health data from a plurality of data collection sources which may include user wearable biosensors, user surveys input via mobile devices, optional user blood testing, and other user data inputs including self-images of the user taken via built in cameras in mobile devices.

In an embodiment, the user wearable biosensors may include smart watches, heart monitors, blood pressure sensors, heart rate sensors, glucose level sensors, and other biometric sensor devices which capture data indicative of a user's health status in real time, or substantially in real-time.

In another embodiment, the system, method, and apparatus includes a front-end client app executable on the mobile device and on other multi-platform front-ends, and which is configured to execute one or more user surveys for receiving feedback directly from the user.

In another embodiment, the one or more surveys conducted by the front-end client app may include an onboarding survey to conduct an initial health assessment of a user in order to establish a baseline starting point.

In another embodiment, the one or more surveys conducted by the front-end client app may further include a daily progress survey which receives daily input from a user regarding the progress of the user's health in response to a nutraceutical polyphenol supplements and dosage regimen.

In another embodiments, the one or more surveys conducted by the front-end client app may further include an artificial intelligence (“AI”) assessment survey which receives regular input from a user regarding updates on the user's health in response to the nutraceutical polyphenol supplements and dosage regimen.

In another embodiment, the front-end client app executable on the mobile device and other multiplatform front-ends is configured to display one or more progress charts, including a health indicator progress chart showing improvements to a user's health over time.

In another embodiment, the front-end client app executable on the mobile device is operatively integrated with a back-end server which is able to access and receive the data collected by the front-end client app executing on one or more multiplatform front-ends. In another embodiment, the back-end server further comprises storage for user data collected by and uploaded from the front-end client app executing on multiplatform front-ends.

In another embodiments, the back-end server further comprises an artificial intelligence (“AI”)/machine learning (“ML”) inference module for analyzing the user data collected by and uploaded from the front-end client app executing on the mobile device or other multiplatform front-ends. Upon performing the analysis, the AI/ML inference module generates an output which is transmitted back to the front-end client app for display on the user's mobile device or on another multiplatform front-end.

In still another embodiment, the AI/ML inference module is operatively connected to an AI/ML engine which trains the AI/ML using all or some of the data collected via the front-end client app, including regularly updated data based on the daily progress surveys and AI assessment surveys conducted through the front-end client app and uploaded to the back-end storage.

In one embodiment, the AI/ML engine trains AI/ML using a random parameter initialization; or an a priori initialization reconstructed from published scientific literature or software; or a combination thereof.

In another embodiment, the AI/ML engine updates AI/ML using additional data collected via the front-end client app and uploaded to the back-end storage since the last training or update of AI/ML.

In yet another embodiment, the data collected by the client app on the mobile device or other multiplatform front-ends, includes image data of the user, which may include images of a user's skin condition to assess texture and appearance. The images of the user's skin may be indicative of various skin conditions including cellulite, dryness, elastosis, etc. By taking updated images of the user's skin in the same location on a regular basis, the back-end AI/ML inference module is able to infer and assess improvements in the user's skin condition resulting from the nutraceutical polyphenol supplements and dosage regimen.

In yet another embodiment, the present system, method, and apparatus track a user's health progress overtime, and outputs a prediction on improvements to the user's projected long-term health based on the nutraceutical polyphenol supplements and dosage regimen. These projected improvements to the user's long-term health may be displayed via the display screen on the mobile device or other multiplatform front-end to show the efficacy of the nutraceutical polyphenol supplements and dosage regimen.

In yet another embodiment, the present system, method, and apparatus tracks user's health data and generates warnings if the levels of data readouts deviate from a specified range of acceptable values.

In yet another embodiment, the present system, method, and apparatus tracks a group of users by anonymizing their data, to show the efficacy of the nutraceutical polyphenol supplements and dosage regimen for a group of users.

In yet another embodiment, the present system, method, and apparatus learns from the data collected for a group of users, and projects the long-term health improvements for a group of users taking the nutraceutical polyphenol supplements and dosage regimen.

In still another embodiment, the present system, method, and apparatus are configured to provide feedback to the user on a change in the nutraceutical polyphenol supplements and dosage regimen, for example a change in the dosage, frequency, or recommending a change in the nutraceutical polyphenol supplements and dosage regimen, based on the projected long-term health of a group of users to fine-tune the health benefits of the nutraceutical polyphenol supplements and dosage regimen.

In this respect, before explaining at least one embodiment of the invention in detail, it is to be understood that the invention is not limited in its application to the details of construction and to the arrangements of the components set forth in the following description or the examples provided therein or illustrated in the drawings. Therefore, it will be appreciated that a number of variants and modifications can be made without departing from the teachings of the disclosure as a whole. Therefore, the present apparatus, system, and method is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

The presence system, method and apparatus, and objects of the invention will become apparent when consideration is given to the following detailed description thereof. Such description makes reference to the annexed drawings, wherein:

FIG. 1 shows a schematic block diagram of a system, method and apparatus in accordance with an illustrative embodiment.

FIG. 2 shows a schematic block diagram of an AI/App backend architecture in accordance with an illustrative embodiment.

FIGS. 3A-3L show screen shots of a front-end client app executing on a mobile computing device for conducting an onboarding survey in accordance with an illustrative embodiment.

FIGS. 4A-4M show screenshots of the front-end client app executing on a mobile computing device with an illustrative App dashboard in accordance with an illustrative embodiment.

FIGS. 5A-5G show screenshots of the front-end client app executing on a mobile computing device for conducting a daily progress survey in accordance with an illustrative embodiment.

FIGS. 6A-6D show screenshots of the front-end client app executing on a mobile computing device for tracking and displaying a user profile in accordance with an illustrative embodiment.

FIGS. 7A-7Z show screenshots of the front-end client app executing on a mobile computing device for performing an AI assessment of efficacy in accordance with an illustrative embodiment.

FIG. 8 shows an illustrative block diagram of a subsystem and method for performing an AI assessment of efficacy utilizing photographic inputs of a user's skin in accordance with an illustrative embodiment.

FIG. 9 shows an illustrative block diagram of a subsystem and method for performing AI based predictions of a user's long-term health based on the user's collected health data, and daily progress updates and AI assessment surveys conducted via the front-end App.

FIG. 10 shows an illustrative block diagram of various accessory devices for collecting user health data in accordance with various embodiments.

FIG. 11 shows an illustrative block diagram of a generic computing system which may provide a platform for various embodiments of the present system, method and apparatus.

In the drawings, embodiments are illustrated by way of example. It is to be expressly understood that the description and drawings are only for the purpose of illustration and as an aid to understanding and are not intended as describing the accurate performance and behavior of embodiments and a definition of the limits of the invention.

### DETAILED DESCRIPTION

As noted above, the present disclosure relates to a system method and apparatus for assessing the efficacy of nutraceutical polyphenol supplements and dosage regimen.

As used in this disclosure, polyphenol supplements may include different types of polyphenols including flavonoids, stilbenes, lignins, lignans, phenolic acids, coumarin, procyanidin, polyphenolic amides, and other polyphenols including but not limited to anthocyanins, resveratrol, ellagic acid, ellagitannins, gallotannins, tannins, quercetin, anthocyanidins, proanthocyanidins, flavones, f flavonols, flavanols, flavanones, isoflavones, hydroxybenzoic acids, hydroxycinnamic acids, catechins, gingerol, cocoa powder, turmeric and curcumin. These polyphenols may be sourced from various plant, food, and pharmaceutical sources, and may be prepared in a natural or concentrated form. These polyphenols may also be blended into proprietary polyphenol formulations, and may be offered in various dosage forms including but not limited to capsules, tablets, pills, drops, and creams.

In an aspect, there is provided a system, method, and apparatus for collection of user health data from a plurality of data collection sources which may include user wearable biosensors, user surveys input via mobile devices, optional user blood testing, and other user data inputs including self-images of the user taken via built in cameras in mobile devices.

In an embodiment, the user wearable biosensors may include smart watches, heart monitors, blood pressure sensors, heart rate sensors, glucose level sensors, and other biometric sensor devices which capture data indicative of a user's health status in real time, or substantially in real-time.

In another embodiment, the system, method, and apparatus includes a front-end client app executable on the mobile device and on other multi-platform front-ends, and which is configured to execute one or more user surveys for receiving feedback directly from the user.

In another embodiment, the one or more surveys conducted by the front-end client app may include an onboarding survey to conduct an initial health assessment of a user in order to establish a baseline starting point.

In another embodiment, the one or more surveys conducted by the front-end client app may further include a daily progress survey which receives daily input from a user regarding the progress of the user's health in response to a nutraceutical polyphenol supplements and dosage regimen.

In another embodiments, the one or more surveys conducted by the front-end client app may further include an artificial intelligence (“AI”) assessment survey which receives regular input from a user regarding updates on the user's health in response to the nutraceutical polyphenol supplements and dosage regimen.

In another embodiment, the front-end client app executable on the mobile device and other multiplatform front-ends is configured to display one or more progress charts, including a health indicator progress chart showing improvements to a user's health over time.

In another embodiment, the front-end client app executable on the mobile device is operatively integrated with a back-end server which is able to access and receive the data collected by the front-end client app executing on one or more multiplatform front-ends. The backend server may comprise one or more of dedicated physical servers or virtual private (virtual dedicated) servers.

In another embodiment, the back-end server further comprises storage for user data collected by and uploaded from the front-end client app executing on multiplatform front-ends.

In another embodiments, the back-end server further comprises an artificial intelligence (“AI”)/machine learning (“ML”) inference module for analyzing the user data collected by and uploaded from the front-end client app executing on the mobile device or other multiplatform front-ends. Upon performing the analysis, the AI/ML inference module generates an output which is transmitted back to the front-end client app for display on the user's mobile device or on another multiplatform front-end.

In still another embodiment, the AI/ML inference module is operatively connected to an AI/ML engine which trains the AI/ML using all or some of data collected via the front-end client app, including regularly updated data based on the daily progress surveys and

AI assessment surveys conducted through the front-end client app and uploaded to the back-end storage.

In one embodiment, the AI/ML engine trains AI/ML using a random parameter initialization; or an a priori initialization reconstructed from published scientific literature or software; or a combination thereof.

In another embodiment, the AI/ML engine updates AI/ML using additional data collected via the front-end client app and uploaded to the back-end storage since the last training or update of AI/ML.

In yet another embodiment, the data collected by the client app on the mobile device or other multiplatform front-ends, includes image data of the user, which may include images of a user's skin condition to assess texture and appearance. The images of the user's skin may be indicative of various skin conditions including cellulite, dryness, elastosis, etc. By taking updated images of the user's skin in the same location on a regular basis, the back-end AI/ML inference module is able to infer and assess improvements in the user's skin condition resulting from the nutraceutical polyphenol supplements and dosage regimen.

In yet another embodiment, the present system, method, and apparatus track a user's health progress overtime, and outputs a prediction on improvements to the user's projected long-term health based on the nutraceutical polyphenol supplements and dosage regimen. These projected improvements to the user's long-term health may be displayed via the display screen on the mobile device or other multiplatform front-end to show the efficacy of the nutraceutical polyphenol supplements and dosage regimen.

In yet another embodiment, the present system, method, and apparatus tracks user's health data and generates warnings if the levels of data readouts deviate from a specified range of acceptable values. Such range may be determined using information retrieved from medical literature solely or on in combination with predictions of AI/ML models made using patterns detected in user's demographic and health data collected by the app, wearable biosensors, weight scales, user surveys input via mobile devices, user blood testing, and other user data inputs collected by the app.

In yet another embodiment, the present system, method, and apparatus tracks a group of users by anonymizing their data, to show the efficacy of the nutraceutical polyphenol supplements and dosage regimen for a group of users.

In yet another embodiment, the present system, method, and apparatus learns from the data collected for a group of users, and projects the long-term health improvements for a group of users taking the nutraceutical polyphenol supplements and dosage regimen.

In still another embodiment, the present system, method, and apparatus are configured to provide feedback to the user on a change in the nutraceutical polyphenol supplements and dosage regimen, for example a change in the dosage, frequency, or recommending a change in the nutraceutical polyphenol supplements or the dosage regimen, based on the projected long-term health of a group of users to fine-tune the health benefits of the nutraceutical polyphenol supplements and dosage regimen. Such feedback may be provided by AI/ML models trained to determine the effectiveness of polyphenol supplements for containment of biomarkers within individually acceptable ranges, or for predicting the risks of current or future diseases or health-related outcomes for multiple, disparate nutraceutical polyphenol supplements and dosage regimen.

Illustrative embodiments will now be described with reference to the drawings.

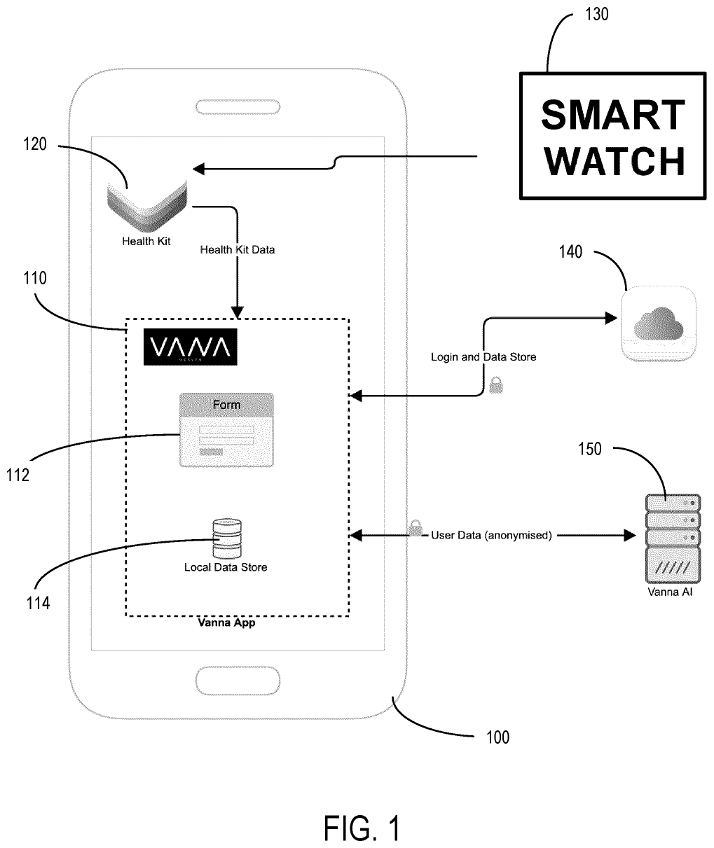

Referring to FIG. 1, shown is a schematic block diagram of a system, method and apparatus in accordance with an illustrative embodiment. As shown, this illustrative example includes a mobile device 100, such as a smart phone, having a memory, storage, and microprocessor configured to execute a front-end client app in accordance with an illustrative embodiment. Here, the client app 110 is shown schematically as a dashed rectangular box, and within the box are illustrative forms 112 with fields receiving user inputs, and schematic local data stores 114 in the mobile device's storage and temporarily in memory.

Still referring to FIG. 1, shown is a smartwatch 130 which is configured to be worn, for example on a user's wrist, and to collect various types of user data, for example a user's blood pressure, pulse rate, temperature, and other physiological attributes in real time, or substantially real-time. Data collected by the smartwatch is transmitted to a health kit app 120 executing on the mobile device.

In an embodiment, the health kit app 120 is configured to communicate health kit data to the front-end client app, which then processes the health kit data for use locally by the front-end client app, and to for transmission to cloud storage 140 for secure storage and further processing.

In another embodiment, data collected by the front-end client app 110 is also transmitted in an anonymized form to the backend AI/ML server 150 for further processing by the AI/ML back-end engine.

While an illustrative example has been shown, it will be appreciated that the system, method, and apparatus may include various other types of sensors to collect user data in real time or virtually real time, and to collect additional user data, automatically or as input by the user, as will be described in further detail below.

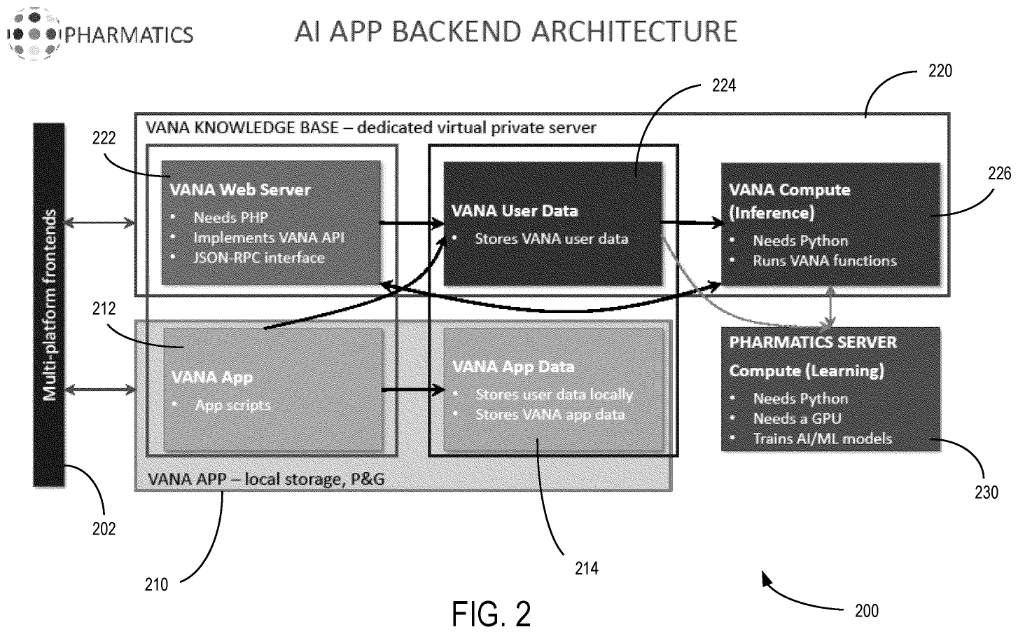

Now referring to FIG. 2, shown is a schematic block diagram of an AI/App backend architecture 200 in accordance with an illustrative embodiment. In this illustrative example, shown at the left are multiplatform front-ends 202 which may include, for example, mobile devices as illustrated in FIG. 1. These mobile devices may execute various different operating systems, such as iOS™ or Android™, for example.

Still referring to FIG. 2, in an embodiment, the front-end client app described above with reference to FIG. 1 is represented by a rectangular box 210 encompassing front-end client app scripts 212, and storage 214 for front-end client app data. A back-end web server 222 is also shown schematically, as running on a dedicated physical or virtual private server 220 to serve the front-end client apps 212. User data collected by the front-end client app 212 is also transmitted to the server 220 and securely stored in user data storage 224. The stored data may be encrypted to protect the user data from unauthorized access even if server security is compromised.

Also shown on the dedicated physical or virtual backend private server 220 is an AI/ML inference module 226 which receives relevant data transmitted by the front-end client app 212, and analyses the data to make inferences about the efficacy of the nutraceutical polyphenol supplements and dosage regimen given an initial baseline for a user and progress data that is collected on a regular basis. In an embodiment, the AI/ML inference module 226 returns an output to the back-end web server 222 for transmission back to the front-end client app, which in turn will display the output of the AI/ML inference module to the user. This will be described in more detail further below.

Still referring to FIG. 2, shown is an AI/ML engine back-end 230 which trains the AI/ML models to be used by the AI/ML inference module 226, or updates the AI/ML models based on learning from user data collected via the front-end client app 212.

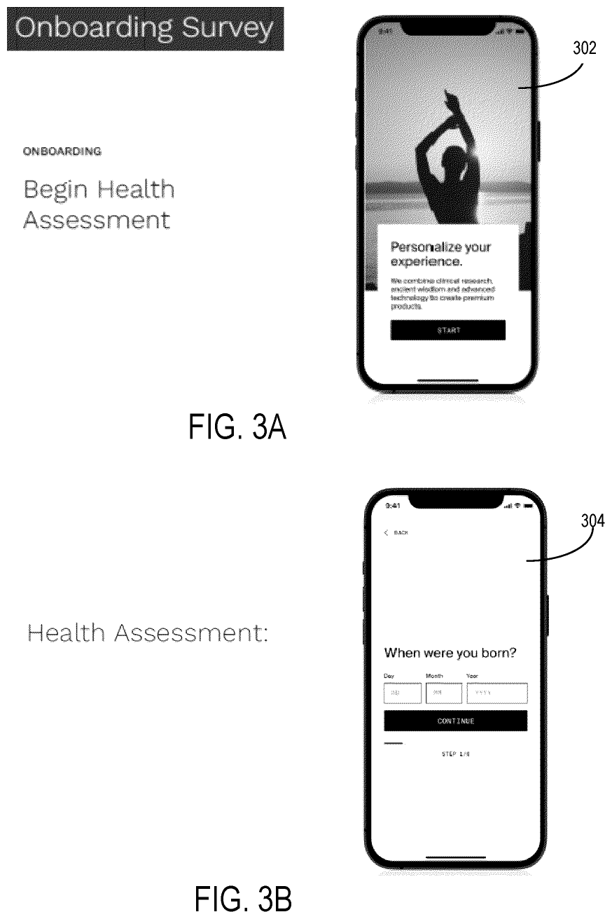

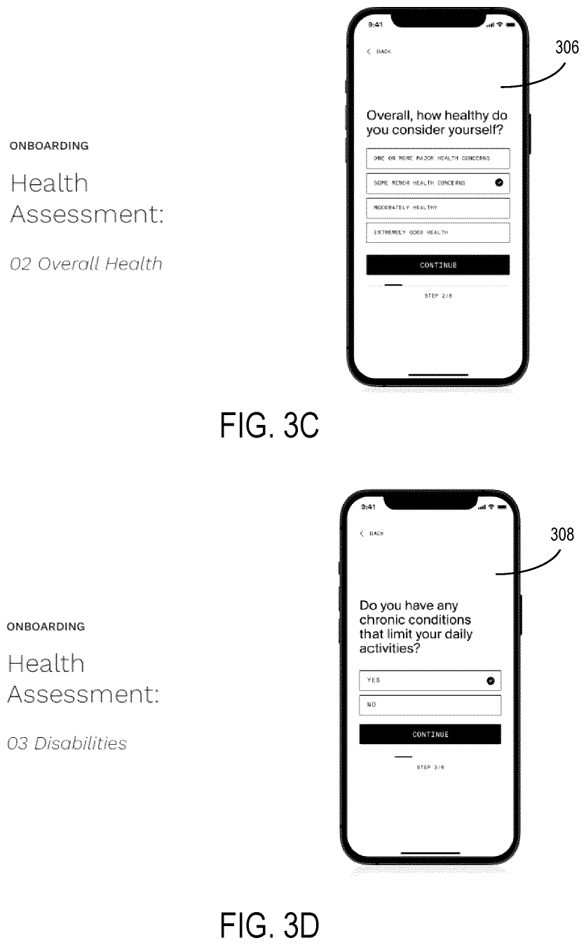

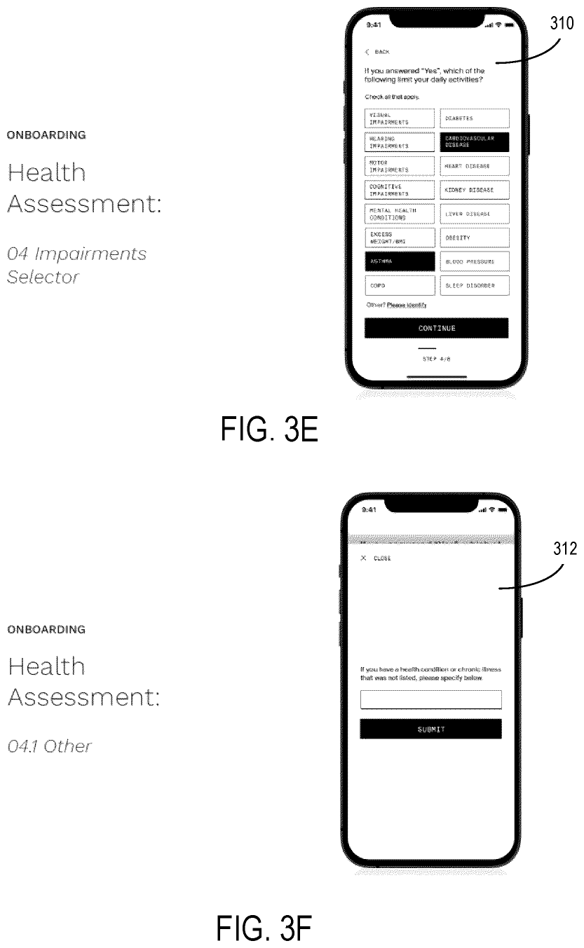

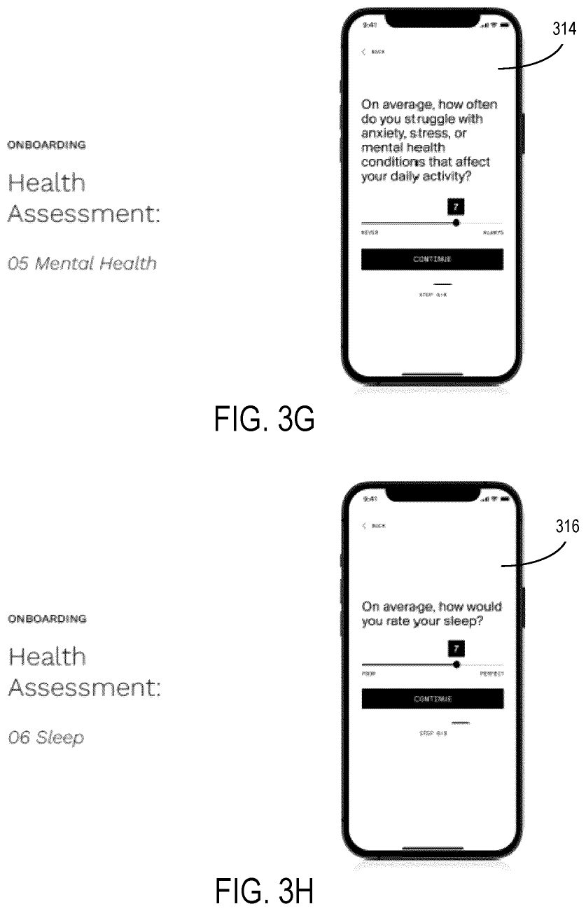

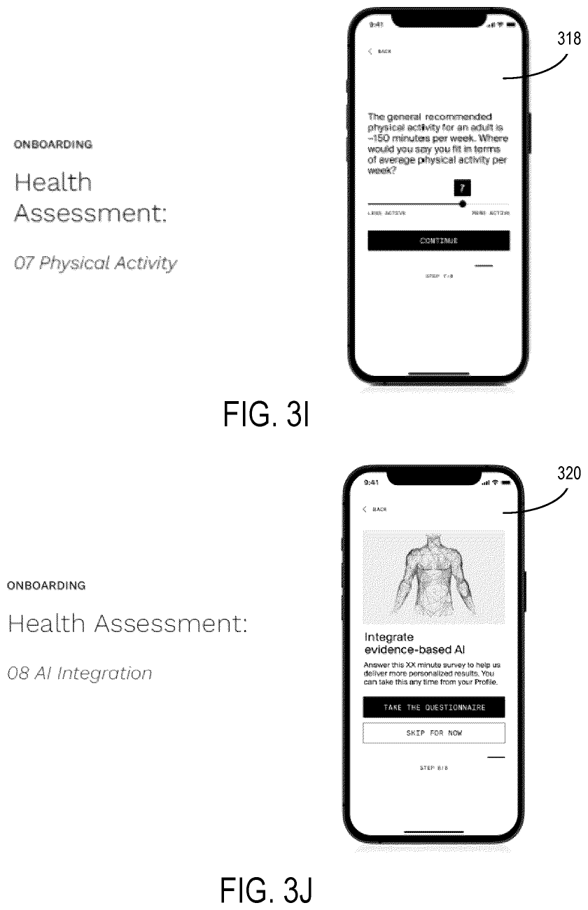

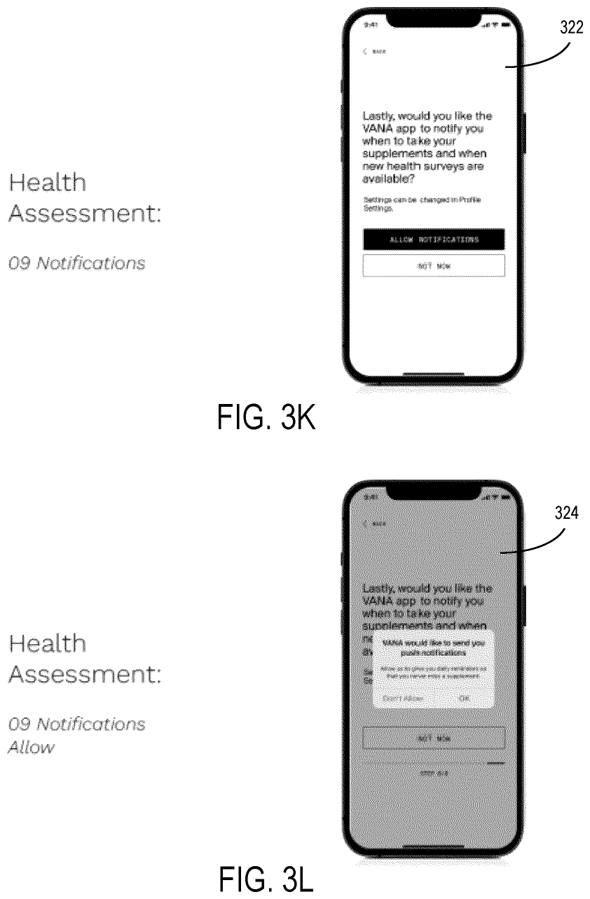

Now referring to FIGS. 3A-3L, shown are screen shots 302-324 of the front-end client app 212 executing on a mobile computing device 100 for conducting an onboarding survey in accordance with an illustrative embodiment.

Starting with FIG. 3A, the front-end App begins an onboarding health assessment survey in order to establish a baseline for a given user. The onboarding health assessment may include age (screen shot 304 of FIG. 3B), overall health as subjectively determined by the user (screen shot 306 of FIG. 3C), any disabilities or chronic conditions that may limit the user's daily activities (screen shot 308 of FIG. 3D) including a selection of impairments to choose from (screen shot 310 of FIG. 3E), or other types of impairments not listed (screen shot 312 of FIG. 3F). The onboarding health assessment may further request an input from the user on a subjective level of anxiety, stress, or mental health conditions that affect a user's daily activities (screen shot 314 of FIG. 3G), and a subjective user rating on the quality of the user's sleep (screen shot 316 of FIG. 3H). The onboarding health assessment may also enquire about the level of physical activity of a user, relative to the average general recommended physical activity for an adult—for example 150 minutes per week (screen shot 318 of FIG. 3I).

Now referring to screen shot 320 of FIG. 3J, the onboarding health assessment may further include AI integration and a questionnaire if AI integration is selected by the user. This AI assessment questionnaire will be described in more detail further below.

The front-end client app may also be configured to provide push notifications to remind the user when to take a dose of the nutraceutical polyphenol supplements, and when new health surveys are available to assess the efficacy of the nutraceutical polyphenol supplements and dosage regimen (screen shot 322 of FIG. 3K). Permission to allow notifications may be granted by the user (screen shot 324 of FIG. 3L).

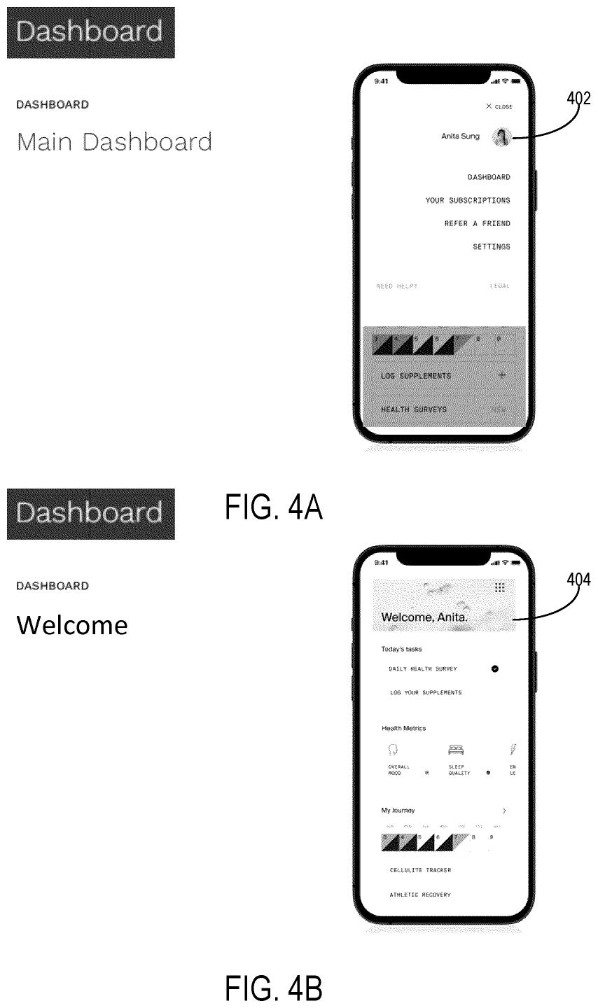

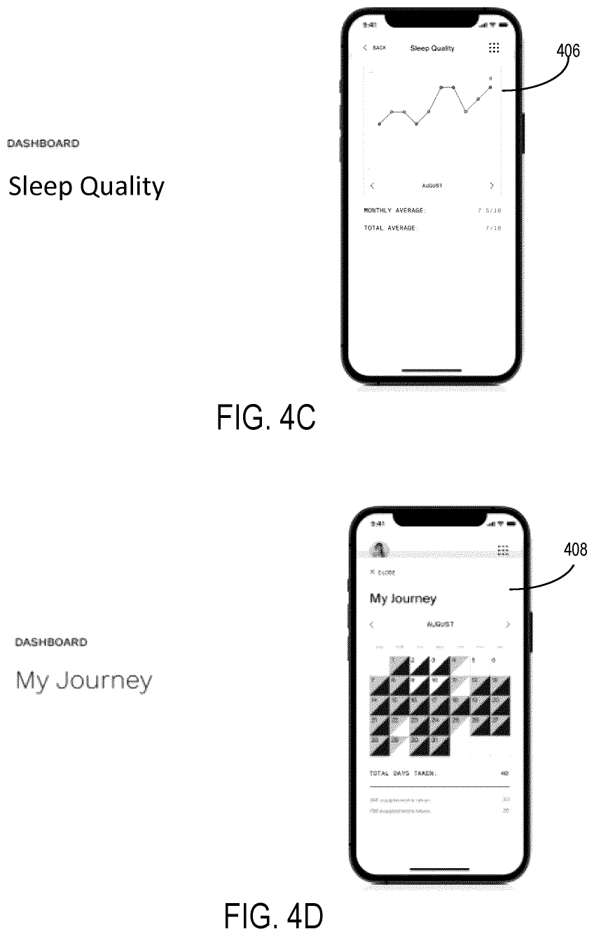

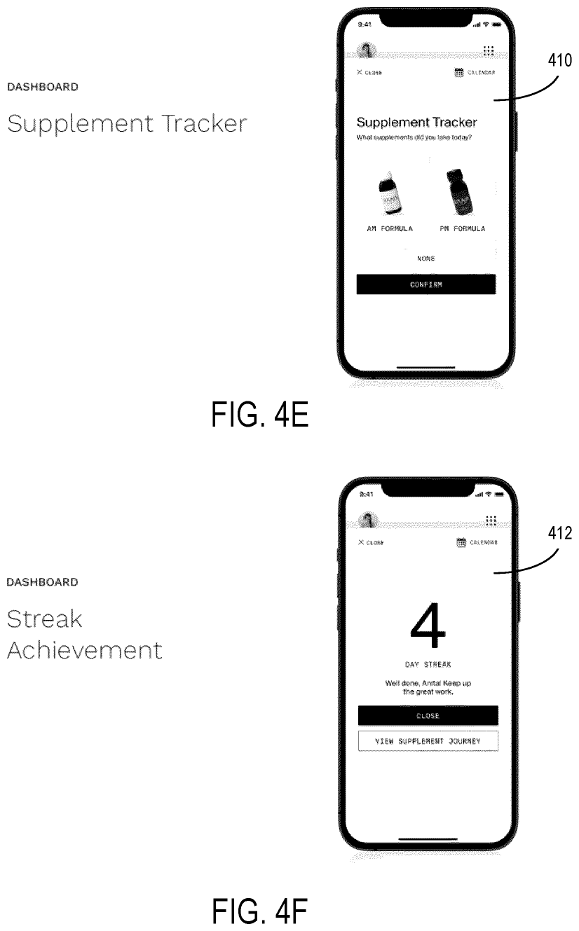

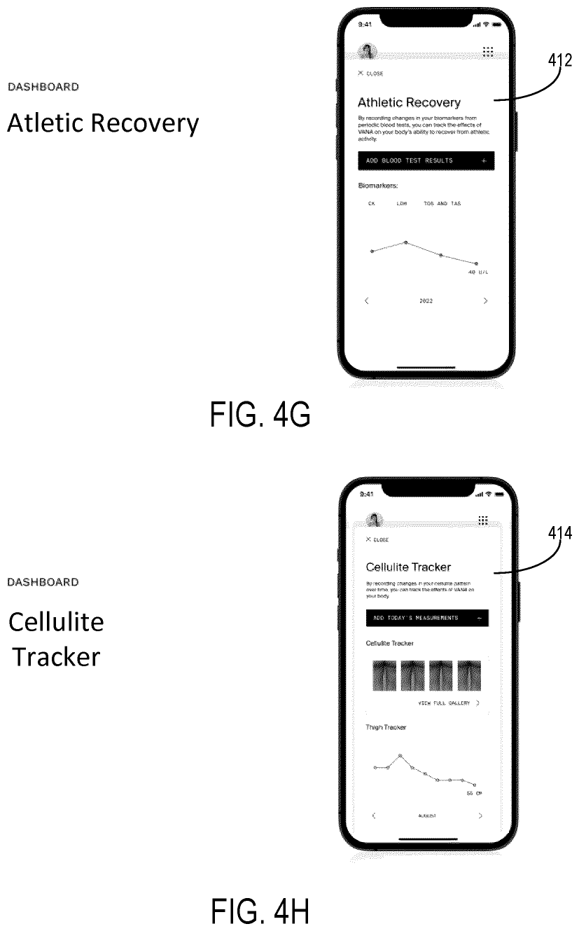

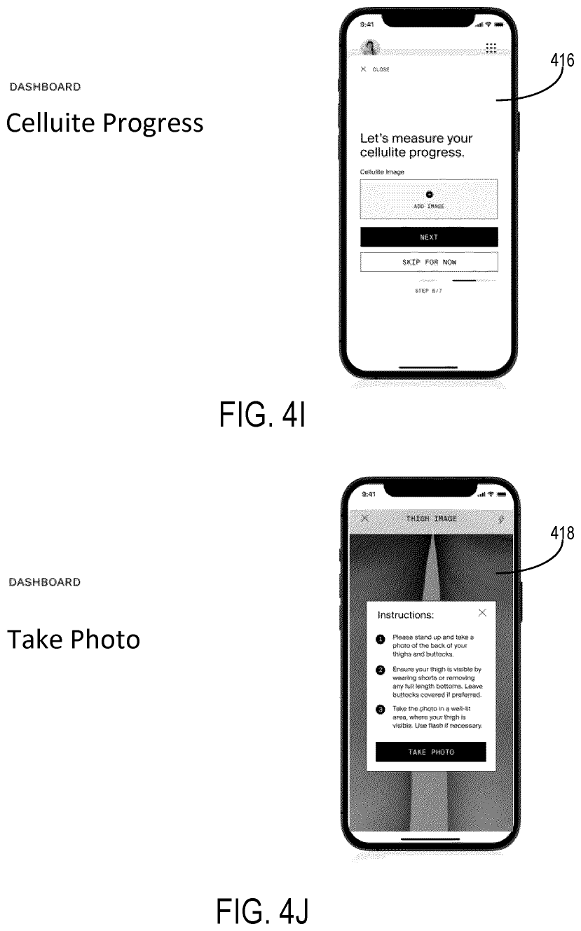

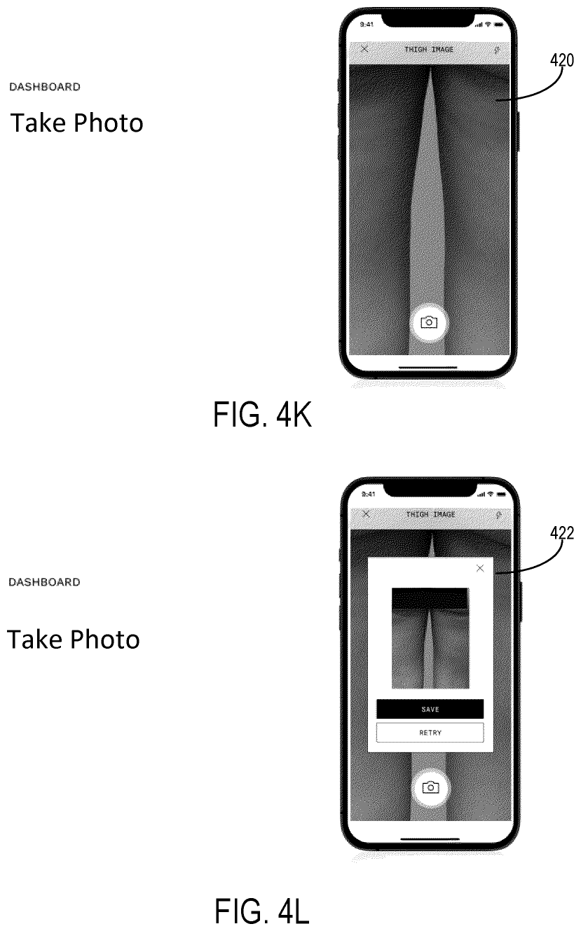

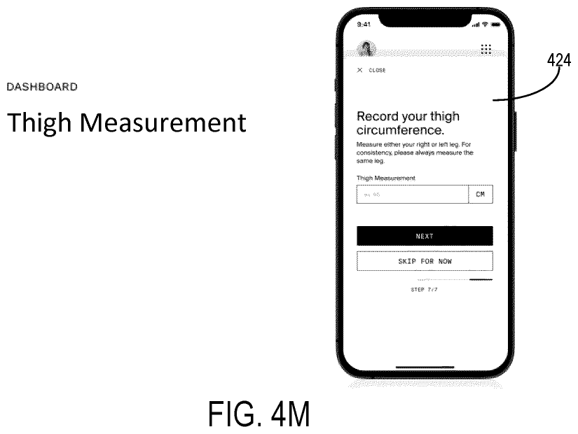

Now referring to FIGS. 4A-4M, shown are screen shots 402-424 of the front-end client app 212 executing on a mobile computing device 100 with an illustrative App dashboard in accordance with an illustrative embodiment.

The front-end client app may display a Main Dashboard (screen shot 402 of FIG. 4A) which includes various submenus for selection, and access to a calendar, a user log, and a daily health progress survey.

A monthly view of the calendar can also be shown (screen shot 408 of FIG. 4D) in order to provide an overview of the nutraceutical polyphenol supplements and dosage regimen completed or to be followed by the user. A user welcome screen (screen shot 404 of FIG. 4B) may present a user with daily updates to a dashboard, including reminders for a daily health survey, a reminder to log supplements, and a dashboard of health metrics, such as overall mood, and sleep quality, which is logged over an extended period of time, as illustrated by way of example in screen shot 406 of FIG. 4C. For assistance with dosage tracking, the front-end App may display also provide user selectable buttons to confirm when a dosage has been taken (screen shot 410 of FIG. 4E). Upon selection of an appropriate button, the App may add the dosage to the count, and indicate that the dosage has been taken in the calendar (screen shot 408 of FIG. 4D). The front-end App may also display continuous streaks (screen shot 412 of FIG. 4F) to show when the user is continuously following the nutraceutical polyphenol supplements and dosage regimen.

In another embodiment, the system may also track the impact of supplements on athletic recovery, as shown by way of example in screen shot 412 of FIG. 4G.

In yet another embodiment, the system may track a user's cellulite by recording changes in a user's cellulite pattern over a period of time, as shown by way of example in screen shot 414 of FIG. 4H. Screen shot 416 of FIG. 4I illustrates how cellulite progress may be measured. In a preferred embodiment, periodic photos are taken of the back of a user's thighs, as illustrated in screen shot 418 of FIG. 4J. Screen shot 420 of FIG. 4K shows a photo that has been taken, and screen shot 422 of FIG. 4L prompts the user to either save the photo, or retry. Screen shot 424 of FIG. 4M further prompts the user to measure the circumference of the thigh, as another input into the cellulite tracker.

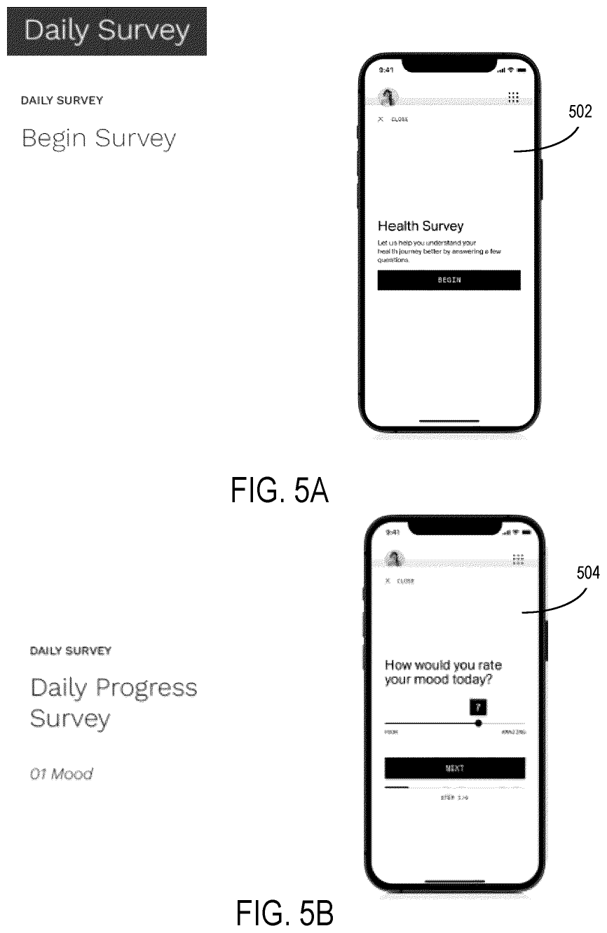

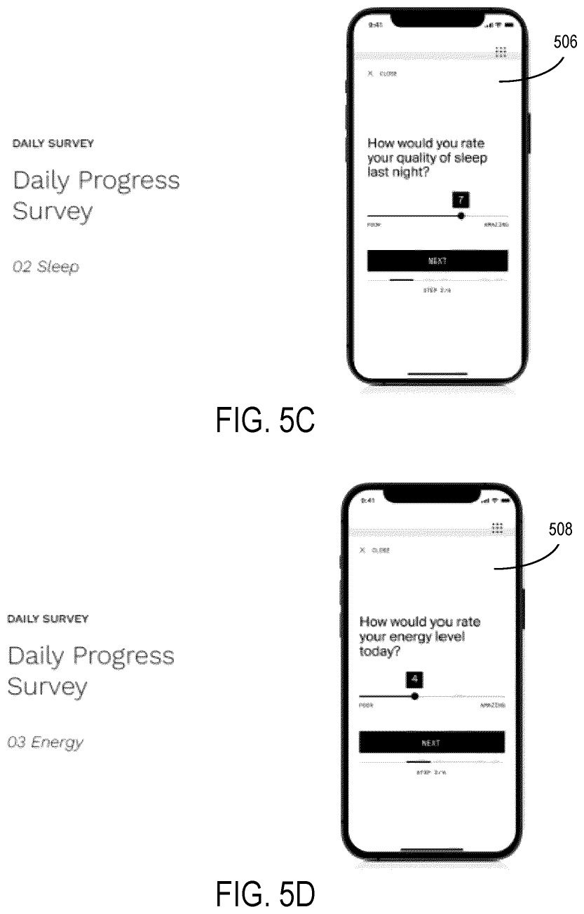

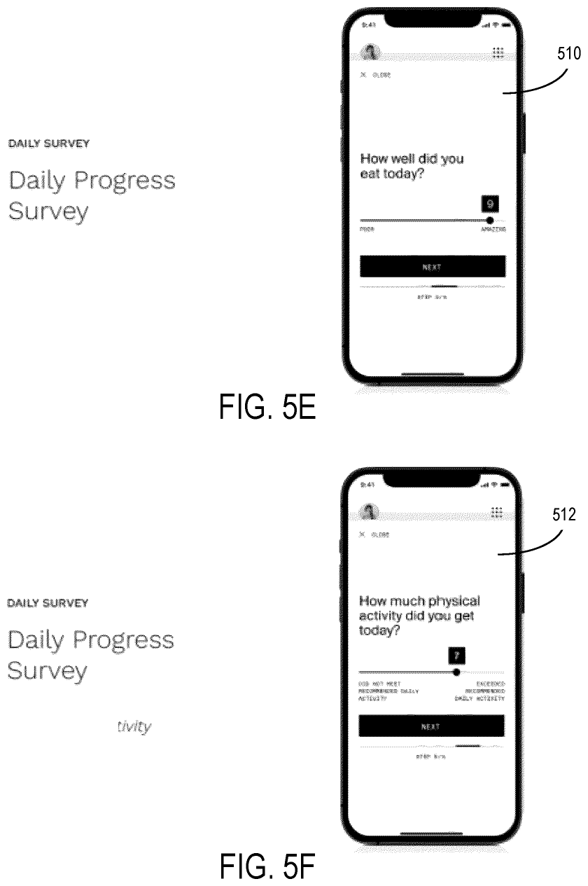

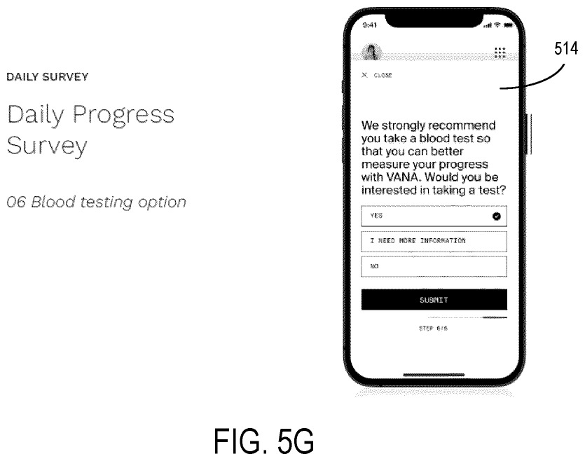

Now referring to FIGS. 5A-5G, shown are screen shots 502-514 of the front-end client app 212 executing on a mobile computing device 100 for conducting a daily progress survey in accordance with an illustrative embodiment.

In this illustrative example (screen shot 502 of FIG. 5A), this optional health survey may be taken by a user on a daily basis to help track the user's progress. The daily progress survey may ask for the user's mood (screen shot 504 of FIG. 5B), and also ask about the user's quality of sleep last night (screen shot 506 of FIG. 5C). The daily survey progress may also ask about the user's energy level (screen shot 508 of FIG. 5D), and also ask how the user ate today (screen shot 510 of FIG. 5E). The daily progress survey may also ask about the amount of physical activity the user has undertaken (screen shot 512 of FIG. 5F).

In an embodiment, the daily progress survey may also ask if the user wishes to take an optional blood test (screen shot 506 of FIG. 5G). While daily blood testing may not be feasible in most cases, this option may be made available on a periodic basis, for example, after three or four months, for those users wishing to track their progress more objectively, using measured levels for various blood properties in order to determine the user's progress over a period of time.

By way of example and not by way of limitation, the blood test could detect and measure the following properties:
HbA1C—Glycated hemoglobin—it shows what the average amount of blood sugar (glucose) attached to hemoglobin has been over the past three months. Screening for type 1 and type 2 diabetes or prediabetes. Prediabetes means your blood sugar levels show you are at risk for getting diabetes.
BUN—Blood Urea Nitrogen—to evaluate the health of your kidneys.
Creatinine—to evaluate the health of your kidneys.
K—Potassium—high levels seen in kidney disease, Addison disease, injury to tissue, infections, diabetes, dehydration and with taking certain medications. Low levels seen with diarrhea and vomiting, Conn syndrome, taking water pills and with certain other medications.
TRIG—Triglycerides-assessing your risk of developing heart disease.
Total Cholesterol—Total Cholesterol in Your Blood-screening for risk of developing heart disease.
HDL—High-Density Lipoprotein-Good Cholesterol-screening for risk of developing heart disease, type-2 diabetes or diabetic complications.
LDL—Low-density Lipoprotein-Bad Cholesterol-screening for risk of developing heart disease or diabetic complications.
hs-CPR—The hs-CRP test accurately measures low levels of CRP to identify low but persistent levels of inflammation and thus helps predict a person's risk of developing CVD, or assess severity of chronic inflammatory disease. These normal but slightly high levels of CRP in otherwise healthy individuals can predict the future risk of a heart attack, stroke, sudden cardiac death, pain interference, and peripheral arterial disease, even when cholesterol levels are within an acceptable range.
Fibrinogen—is a protein, specifically a clotting factor (factor I), that is essential for proper blood clot formation. Blood levels of fibrinogen along with other acute phase reactants rise sharply with conditions causing acute tissue inflammation or damage. It helps determine overall risk of cardiovascular disease.
TNF-α—is an inflammatory cytokine produced by macrophages/monocytes during acute inflammation. Serum or plasma levels may be elevated in sepsis, autoimmune diseases, various infectious diseases, transplant rejection and in patients with suspected chronic inflammatory disorders, such as rheumatoid arthritis, inflammatory bowel disease, ankylosing spondylitis, or cancers.
IL-6—Interleukin-6 is a protein produced by various cells and the test measures the amount of IL-6 in the blood. IL-6 is produced in the body, wherever there is inflammation, either acute or chronic. Used to help monitor inflammatory responses such as infection, sepsis, lupus, or rheumatoid arthritis or to evaluate diabetes, stroke, and cardiovascular disease.
PSA (FOR MEN)—Prostate-specific antigen-checking prostate health, indicates inflammation of the prostate.
Hemoglobin—screening for, diagnosing and measuring the severity of anemia or polycythemia.
Ferritin—A blood protein that contains iron-screens for iron deficiency anemia or higher than normal ferritin can be seen in Hemochromatosis, liver disease, porphyria, rheumatoid arthritis or another chronic inflammatory disorder, hyperthyroidism, leukemia, alcohol abuse, Hodgkin's lymphoma.
Platelet aggregation—Checks how well your platelets clump together to form blood clots-Screening for an improvement in the platelet activity aggregation and activation important for prevention of a clot formation and prevention of atherosclerosis.
D—Dimer—is one of the protein fragments produced when a blood clot gets dissolved in the body. Screening for an improvement in platelet function.
Vitamin K—It is essential for the formation of several substances called coagulation factors as well as protein C and protein S that work together to clot the blood when injuries to blood vessels occur and to prevent excessive clotting. Insufficient vitamin K can lead to excessive bleeding and easy bruising. Vitamin K is also thought to play an important role in the prevention of bone loss. Screening for an improvement in platelet function.
ET-1—Endothelin-1—measures blood vessel elasticity. Elevated plasma concentrations of endothelin's have been observed in hypertension, myocardial infraction, cardiogenic shock, Raynaud syndrome and Crohn's disease.
TOS and TAS—Total oxidant status and total antioxidant status-(TOS and TAS are markers of total oxidative status and show the imbalance between free radicals and antioxidants in your body-high oxidative stress leads to aging and can cause damage to many of your tissues, which can lead to a number of diseases over time).

If a user selects the optional blood work, and receives the results of the blood testing, the user can optionally enter the data into the front-end App for collection and transmission to the back-end Web app. If available, specific measurements from the blood testing could be used by the AI to make predictions on a user's long-term health, as will be described in more detail further below.

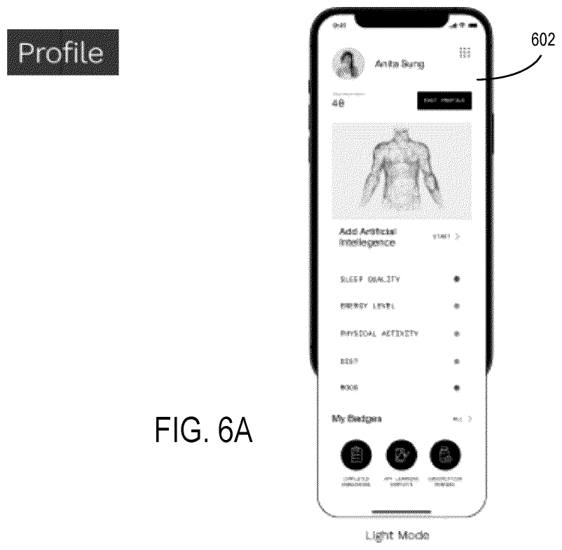

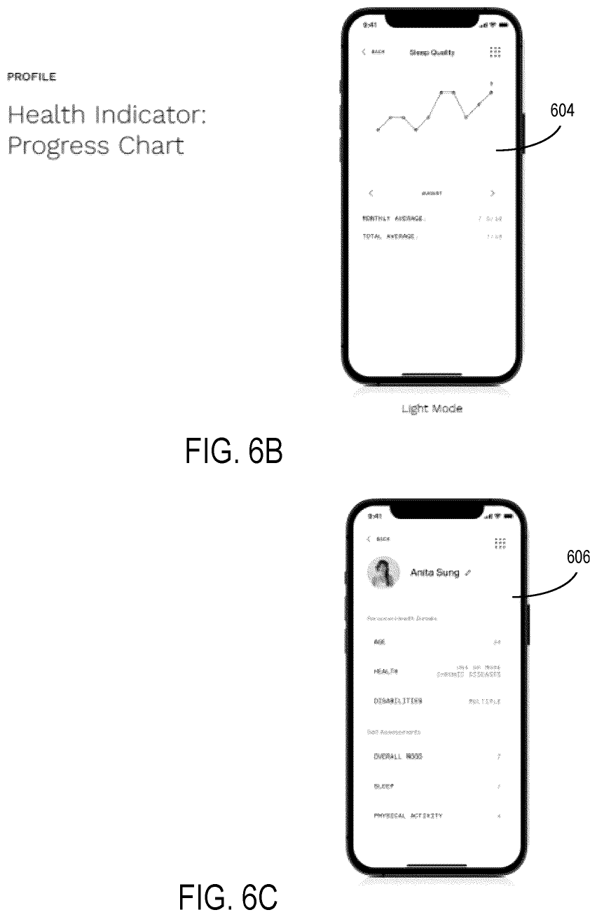

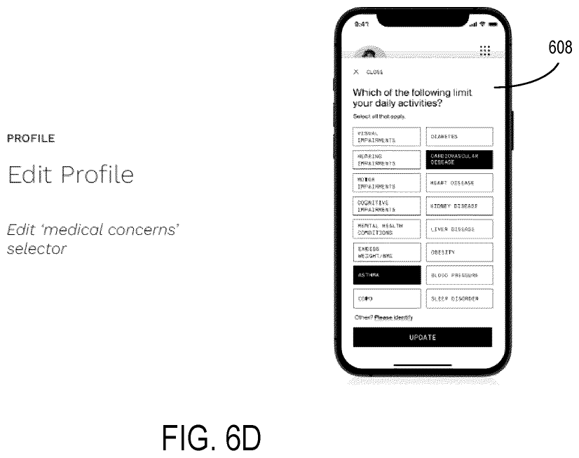

Now referring to FIGS. 6A-6D, shown are screenshots 602-608 of the front-end client app 212 executing on a mobile computing device 100 for tracking and displaying a user profile in accordance with an illustrative embodiment (screen shot 602 of FIG. 6A). By way of example, and not by way of limitation, the user profile may track sleep quality over time, as input by the user (screen shot 604 of FIG. 6B). Various other types of graphs could also be prepared to track other datapoints over time. Personal health details may summarize the data collected by the onboarding survey (screen shot 606 of FIG. 6C). The user may also edit and change data by reviewing previously selected items, such as the “medical concerns” selector shown by way of example in screen shot 608 of FIG. 6D. If certain medical concerns improve and are no longer an issue, the user may also delete these selections.

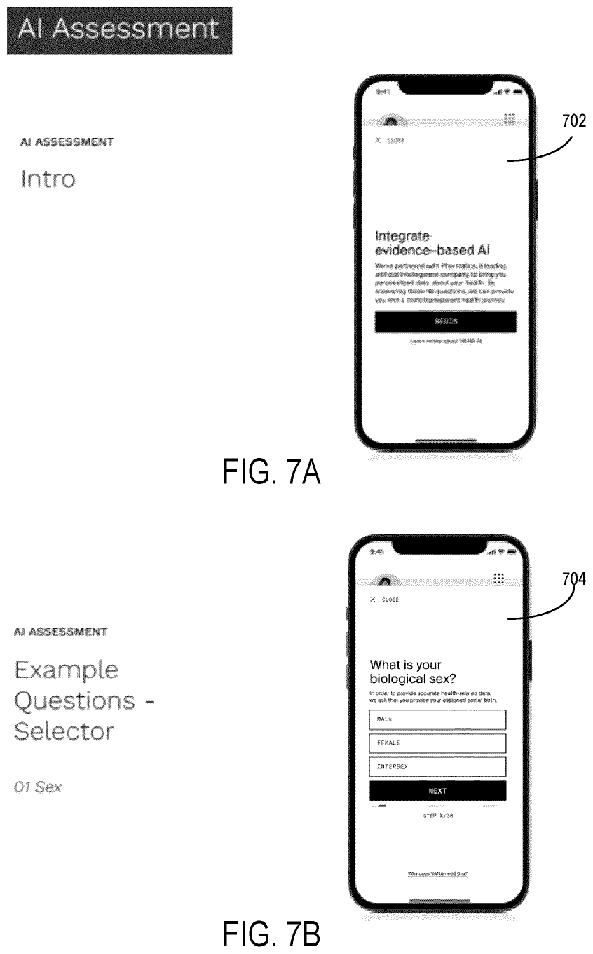

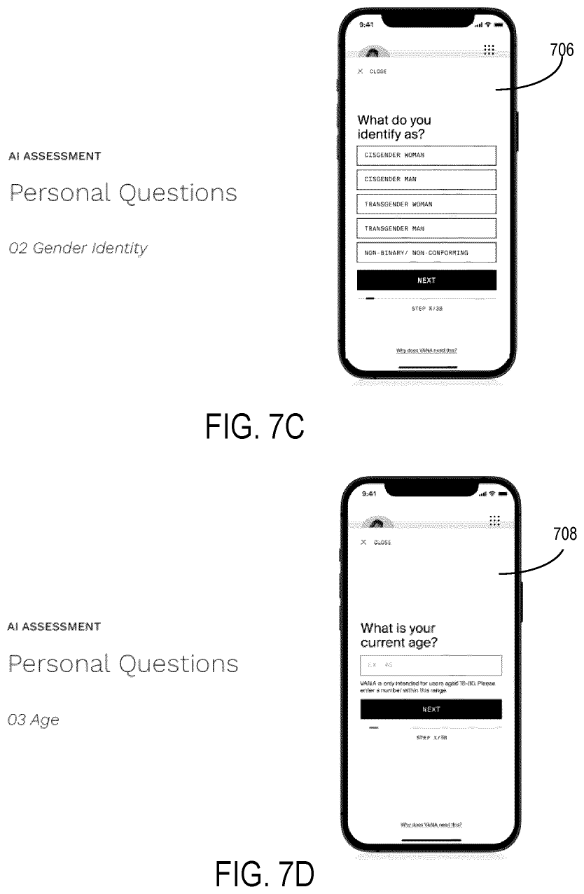

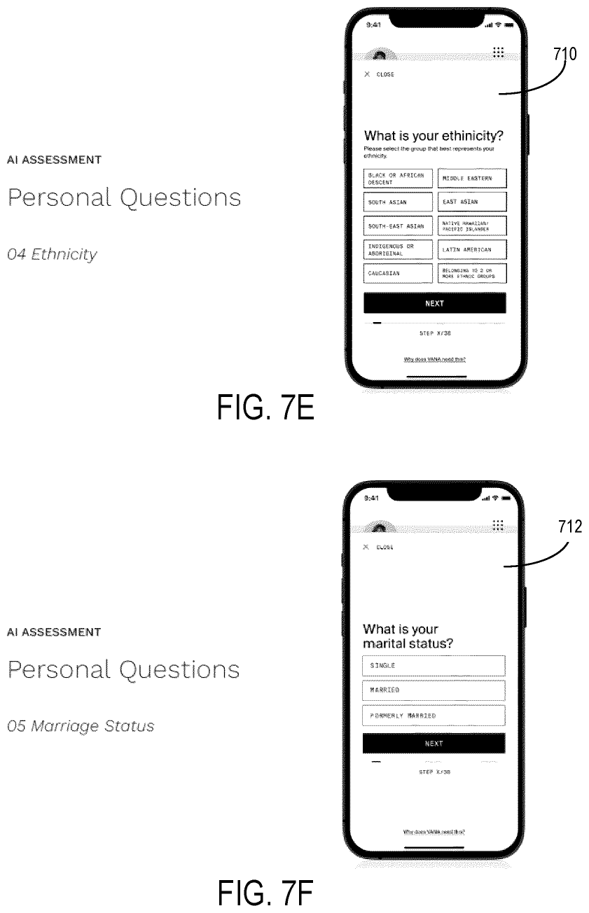

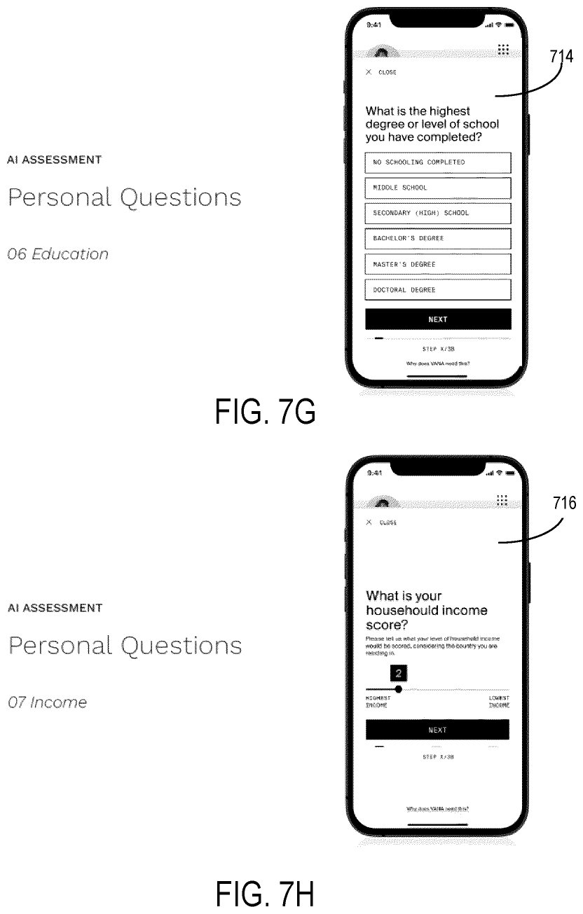

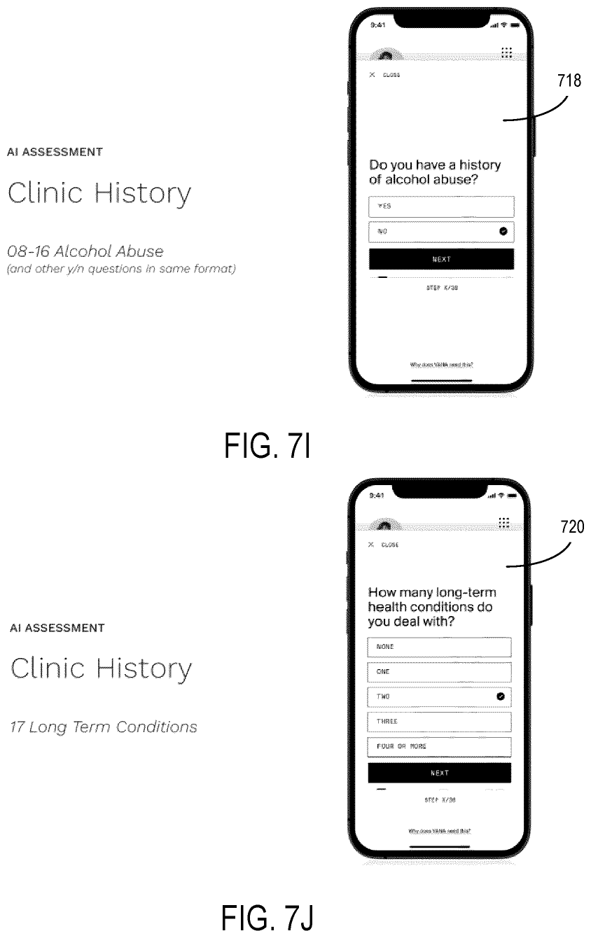

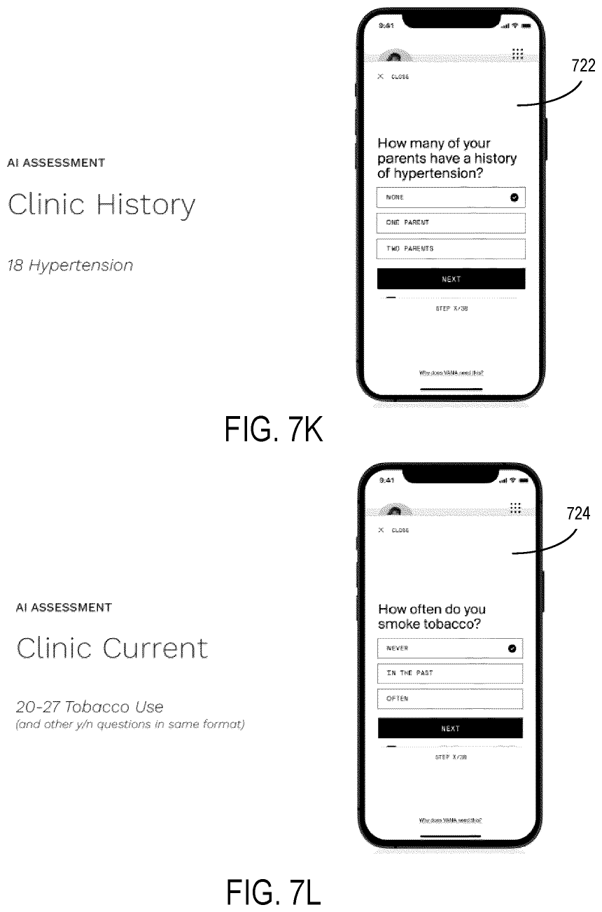

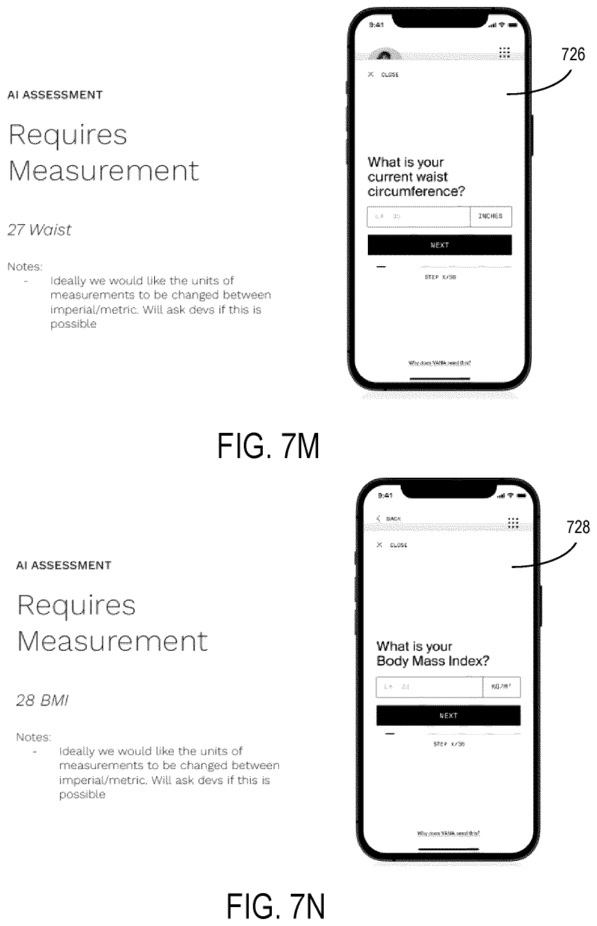

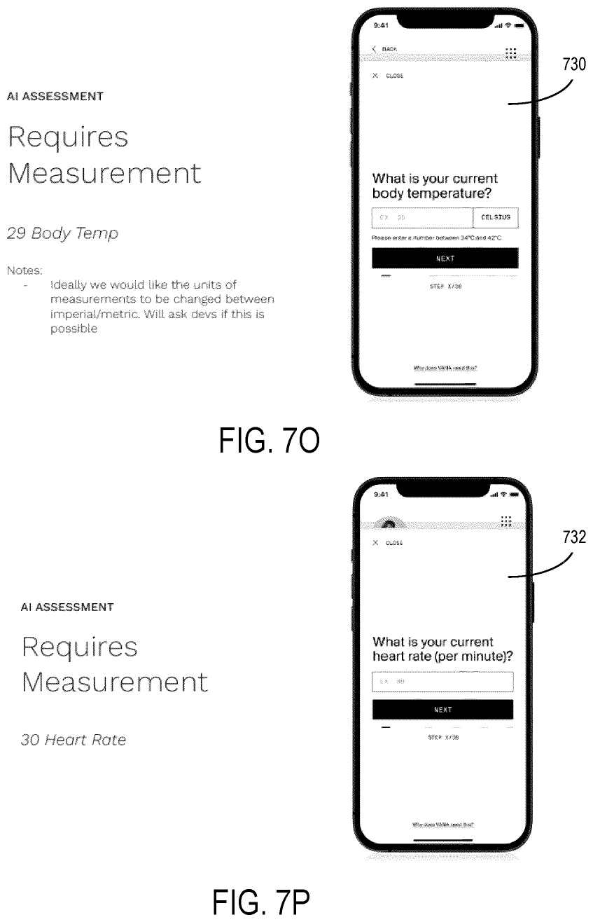

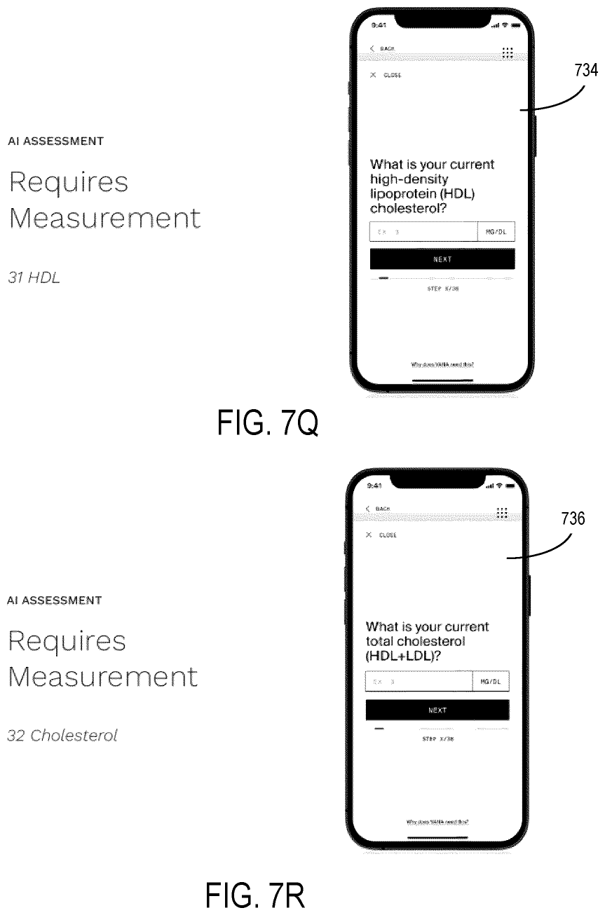

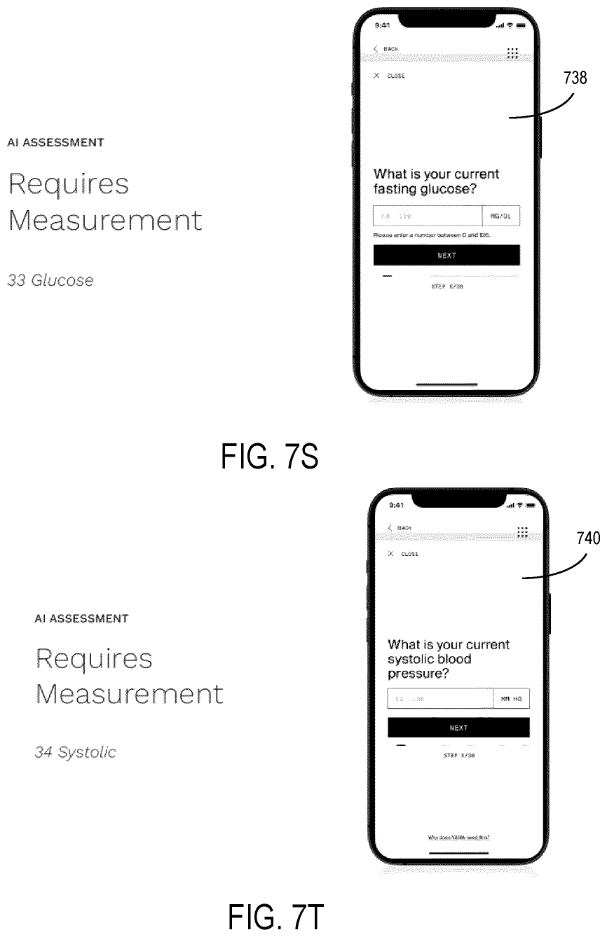

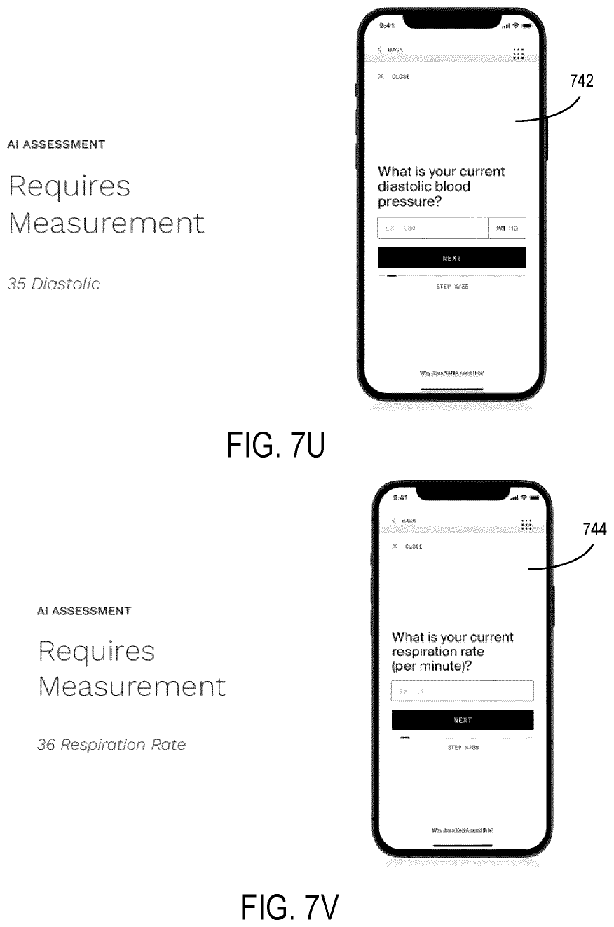

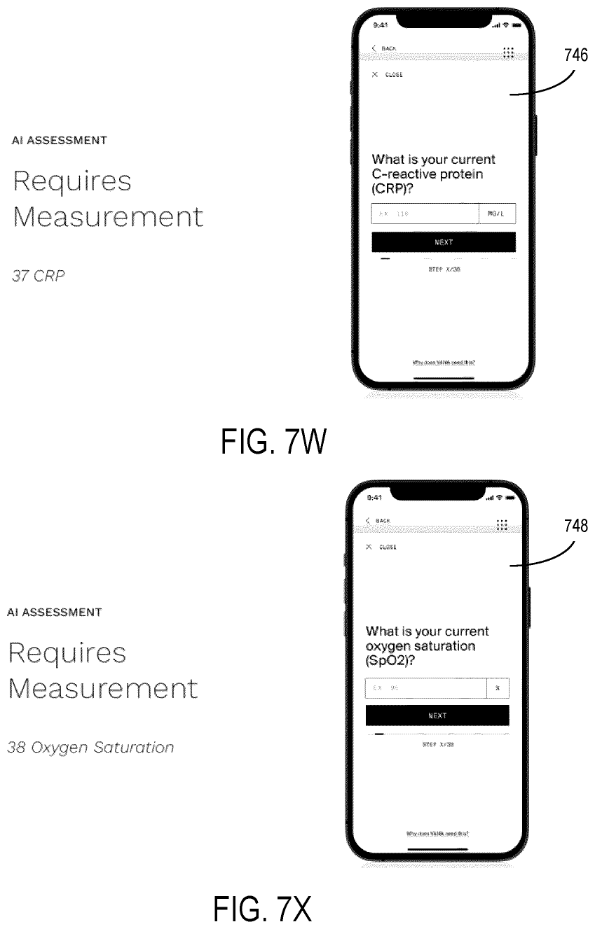

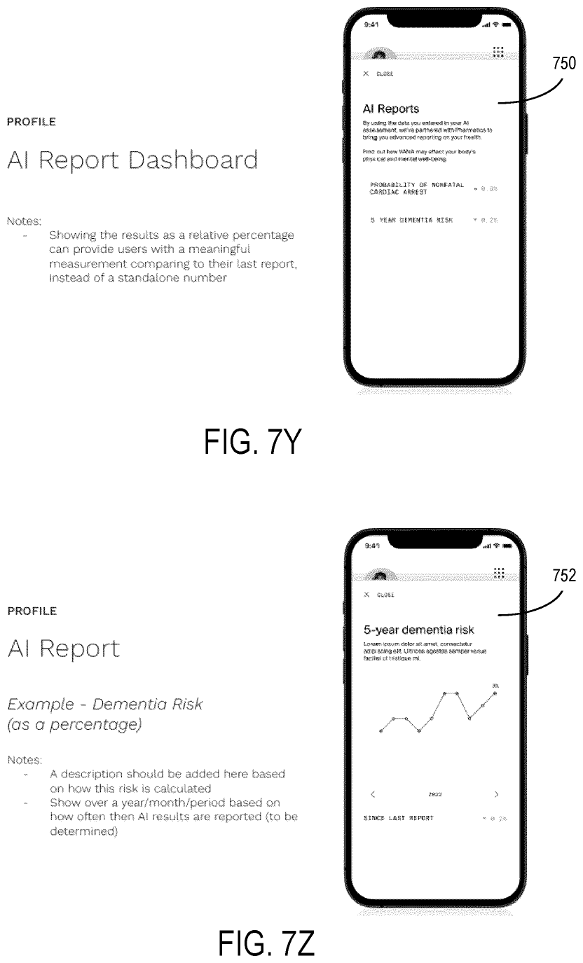

Now referring to FIGS. 7A-7Z, shown are screenshots 702-752 of the front-end client app 212 executing on a mobile computing device 100 for performing an AI assessment of efficacy in accordance with an illustrative embodiment. The introduction page shown in screen shot 702 of FIG. 7A may summarize the way that AI is used by the system, and may also provide a link to a Web page which outlines the process in greater detail. Illustrative examples of questions asked during the AI assessment survey may include the user's personal information including sex, gender identity, age, ethnicity, marriage status, education, and income (screen shots 704-716 of FIGS. 7B-7H). The AI assessment survey may also ask questions relating to clinical history, such as alcohol use, tobacco use, and various long term conditions such as hypertension (screen shots 718-724 of FIGS. 71-7L). The AI assessment survey may also query the user's physiological measurements such as waist size, BMI, body temperature, and heart rate (screen shots 726-732 of FIGS. 7M-7P). The AI assessment survey may query other measurements such as HDL, cholesterol, glucose, systolic and diastolic pressures, and respiration rate (screen shots 734-744 of FIGS. 7Q-7V). Other specific measurements such as the C-reactive protein (CRP) and oxygen saturation level may also be queried (screen shots 746-748 of FIGS. 7W and 7X). Based on the analysis performed by the AI, a report dashboard may show various calculated risks for various conditions, such as illustrated by way of example in screen shots 750-752 of FIGS. 7Y and 7Z.

The list of questions that may be asked during the AI assessment survey is by way of illustration only, and it will be appreciated that the survey may go into much greater detail as may be required by the AI/ML engine to build an AI model with a sufficiently large dataset.

In another embodiment, the AI/ML engine is adapted to have the ability to integrate data collected on other apps and importing it as anonymous data collected for AI analysis. By way of example, data may be sourced from an app that collects daily food intake including your calories, protein, carbs and sugar. Assessing how the type of diet or foods eaten or type and duration of exercise performed effects the benefits of a nutraceutical polyphenol supplements intake may be factored into the analysis performed by the AI/ML engine.

In another embodiment, the AI/ML engine is adapted to assess if the taking of nutraceutical polyphenol supplements and dosage regimen adversely effects or has minimal health benefits for an individual, and could be configured to modify intake or to stop taking it. This may be necessary if a person is allergic to one or more of the ingredients of the nutraceutical polyphenol supplements, for example.

In another embodiment, the AI/ML engine may be adapted to determine whether a person's biomarkers or measurements are at a critical level, and notify them that they should seek medical help/care as soon as possible. Different levels may trigger different levels of warnings depending on how far the levels are from nominal values.

In another embodiment, the AI/ML engine may be adapted to determine whether certain biomarkers or measurements show an improvement in a user's athletic performance, including improvements in recovery after an athletic event. As an example, the system and method may track and create charts for creatinine kinase, lactate dehydrogenase, and total oxidant status (TOS) and total antioxidant status (TAS) biomarkers. These biomarkers may be measured and input to the system and method via an initial onboarding survey as described above, or periodically via daily progress surveys or other periodic inputs.

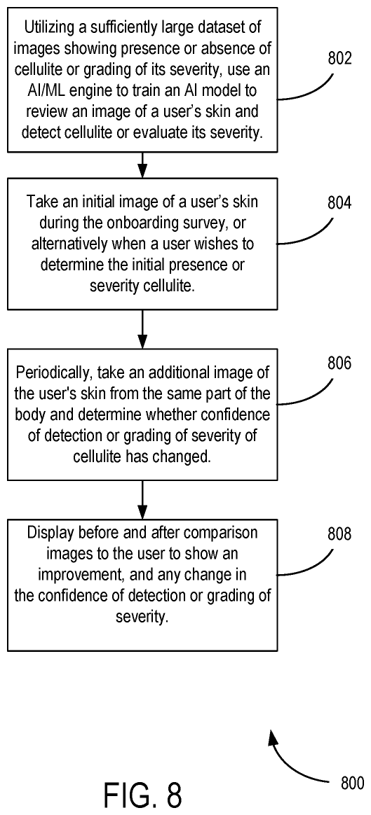

Now referring to FIG. 8, shown is an illustrative block diagram 800 of a subsystem and method for performing an AI assessment of efficacy utilizing photographic inputs of a user's skin in accordance with an illustrative embodiment. In this illustrative example, the system and method performs the following steps:
(i) Block 802: Utilizing a sufficiently large dataset of images showing presence or absence of cellulite or grading of its severity, use an AI/ML engine to train an AI model to review an image of a user's skin and detect cellulite or evaluate its severity.
(ii) Block 804: Take an initial image of a user's skin during the onboarding survey, or alternatively when a user wishes to determine the initial presence or severity cellulite.
(iii) Block 806: Periodically, take an additional image of the user's skin from the same part of the body and determine whether confidence of detection or grading of severity of cellulite has changed.
(iv) Block 808: Display before and after comparison images to the user to show an improvement, and any change in the confidence of detection or grading of severity.

At blocks 804 and 806, the user may choose a specific body part from which to take a photograph, such as the thighs or hips. These photographs are stored in order to compare past photos with more recent photos, and may be shown in a time-lapse sequence of images, or as a gallery of images, to show improvements over a significant period of time which may not be apparent from one photo to the next.

By tracking a user's improvement in physical appearance over time, the AI is able to show in a photo comparison the efficacy of nutraceutical polyphenol supplements and dosage regimen, and including an objective grading of any improvement by the AI.

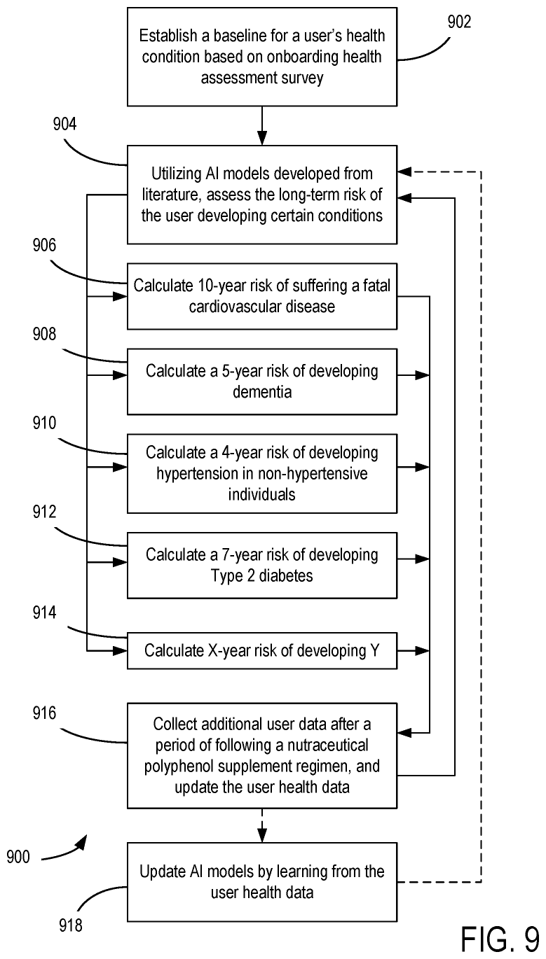

Now referring to FIG. 9, shown is an illustrative block diagram 900 of a subsystem and method for performing AI based predictions of a user's long-term health based on the user's collected health data, and daily progress updates and AI assessment surveys conducted via the front-end client app. By creating AI models based on background research of relevant literature, the present system, method and apparatus is able to infer from the collected user health data the long-term likelihood of a user developing an ailment. As an initial step, at block 902, the system and method may establish a baseline for a user's health condition base on onboarding health assessment survey. At block 904, utilizing AI models developed from literature, assess the long-term risk of the user developing certain conditions. The system and method may then calculate the long-term risk of the user developing a condition, such as a 10-year risk of suffering a fatal cardiovascular disease (block 906), a 5-year risk of developing dementia (block 908), a 4-year risk of developing hypertension in non-hypertensive individuals (block 910), or a 7-year risk of developing Type 2 diabetes (block 912), for example. More generally, at block 914, the system and method may calculate the long-term X-year risk of the user developing a Y-condition.

As an illustration, we present calculation of the risk of developing dementia over time in elderly people (age≥60) based on the Framingham Study of Li et al (2018)¹ modified for a user continuously following the nutraceutical polyphenol supplements and dosage regimen, with the 5-year probability determined according to the following relation: ¹Jinlei Li and Matthew Ogrodnik and Sherral Devine and Sanford Auerbach and Philip A Wolf and Rhoda Au, “Practical risk score for 5-, 10-, and 20-year prediction of dementia inÂ elderly persons: Framingham Heart Study”, Alzheimer's & dementia: the journal of the Alzheimer's Association 14 (1): 35-42, 2018.

$${p}_{t} = 1 - {0.9618}^{s - 0.6572} where$$

$$s = I ( 70 \leq age < 80 ) \times 1.1086 + I ( age \geq 80 ) \times 2.0881 + I ( single ) \times 0.27 + I ( married formerly ) \times 0.174 + I ( BMI < 18.3 ) \times 0.6678 - I ( BMI > 24.8 ) \times 0.1508 + I ( stroke ) \times 0.8198 + I ( diabetes ) \times 0.3507 ++ I ( ischemic attack ) \times 0.6981 + I ( cancer ) \times 0.2852$$

and I(x) is the indicator variable equal to 1 if and only if x is true and equal to zero otherwise.

In an embodiment, after collecting additional user data after a period of following a nutraceutical polyphenol supplements and dosage regimen, the system and method may repeat the step of utilizing AI models developed from literature, to assess the long-term risk of the user developing certain conditions, or track progression of existing conditions, with the new, updated user health data.

In another embodiment, after collecting additional user data after a period of following a nutraceutical polyphenol supplements and dosage regimen, the system and method may update AI models by learning from the user health data by using the models developed from literature as an initialization. Such new updated AI models may be used for assessing the long-term risk of the user developing certain conditions, or for tracking progression of existing conditions, with the new, updated user health data.

Over a period of time, the system and method can track improvements in the predicted long-term health of the user based on following the nutraceutical polyphenol supplements and dosage regimen.

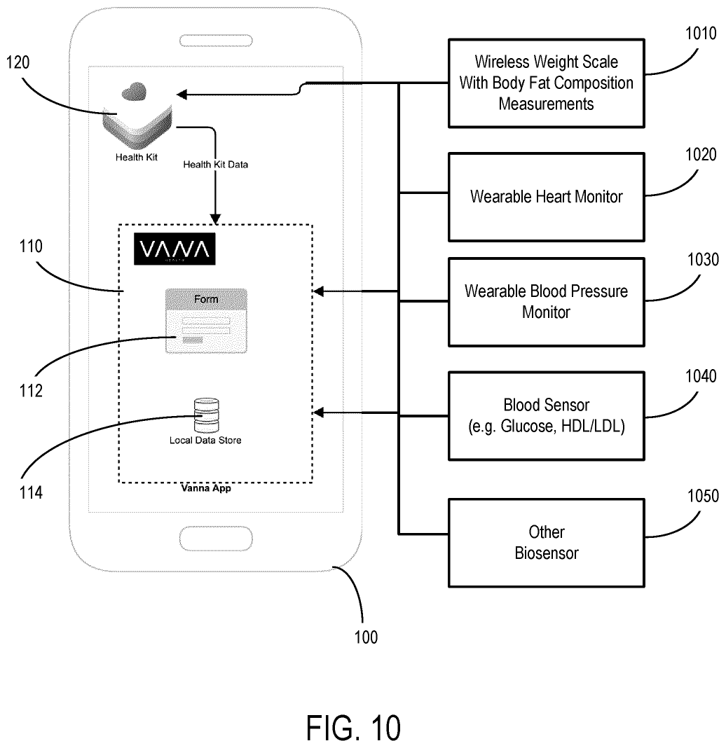

Now referring to FIG. 10, shown is an illustrative block diagram 1000 of various accessory devices for collecting user health data in accordance with various embodiments. These sensors may include, but are not limited to, weight scales 1010 which may also include electronic sensors for detecting body fat composition and other measurements, wearable heart monitor 1020 for monitoring pulse rates, wearable blood pressure monitoring device 1030 for periodically measuring blood pressure, blood sensor 1040 (e.g. to measure glucose levels, or HDL/LDL), and other types of biosensors 1050. Any or all of these accessories may be used to collect and input additional user health data into the front-end client app, either through a wired connection, or wireless connections such as Bluetooth, or Wi-Fi, for example.

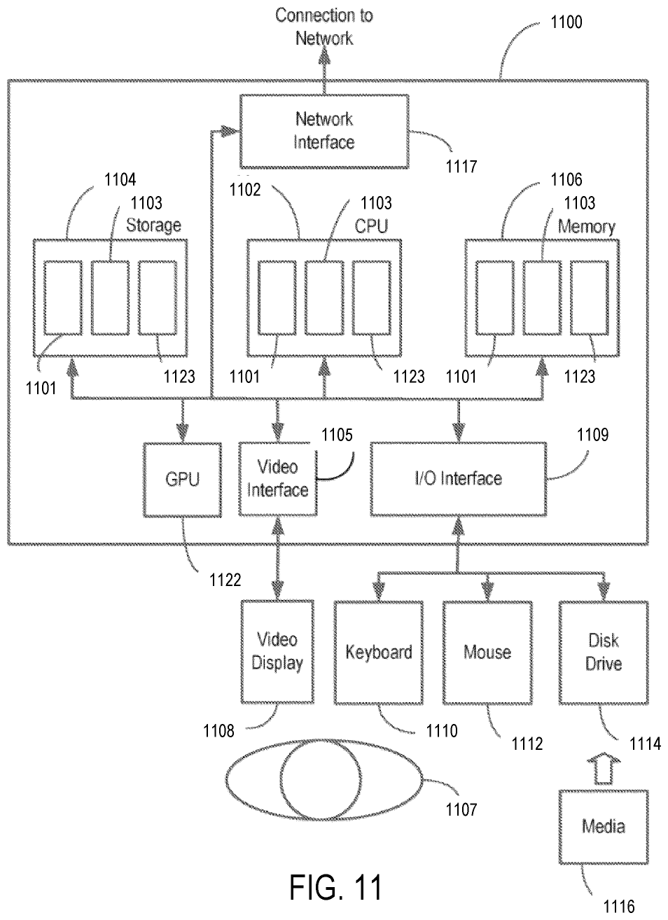

Now referring to FIG. 11 shown is a schematic block diagram of a generic computing device 1100 that may provide a suitable operating environment in one or more embodiments. A suitably configured computer device, and associated communications networks, devices, software and firmware may provide a platform for enabling one or more embodiments as described above. By way of example, FIG. 11 shows a generic computer device 1000 that may include a central processing unit (“CPU”) 1102 connected to a storage unit 1104 and to a random access memory 1106. The CPU 1102 may process an operating system 1101, application program 1103, and data 1123. The operating system 1101, application program 1103, and data 1123 may be stored in storage unit 1104 and loaded into memory 1106, as may be required. Computer device 1100 may further include a graphics processing unit (GPU) 1122 which is operatively connected to CPU 1102 and to memory 1106 to offload intensive calculations (including, but not limited to image processing or inference or training of deep learning AI models) from CPU 1102 and run these calculations in parallel with CPU 1102. An operator 1110 may interact with the computer device 1100 using a video display 1108 connected by a video interface 1105, and various input/output devices such as a keyboard 1110, pointer 1112, and storage 1114 connected by an I/O interface 1109. In known manner, the pointer 1112 may be configured to control movement of a cursor or pointer icon in the video display 1108, and to operate various graphical user interface (GUI) controls appearing in the video display 1108. The computer device 1100 may form part of a network via a network interface 1111, allowing the computer device 1100 to communicate with other suitably configured data processing systems or circuits. A non-transitory medium 1116 may be used to store executable code embodying one or more embodiments of the present method on the generic computing device 1100.

Thus, in an aspect, there is provided a computer-implemented method of assessing the efficacy of a nutraceutical polyphenol supplements and dosage regimen, the method executable on a computing device having a processor, a memory, and storage, and comprising: collecting user health data from one or more biosensors, and from health assessment surveys executed on the computing device to establish a baseline for a user's health condition; collecting data on the nutraceutical polyphenol supplements and dosage regimen a user is taking over time; and monitoring the user's health condition at regular intervals using one or more biosensors and health assessment surveys, and comparing the user's updated health condition against the user's baseline to determine efficacy of the nutraceutical polyphenol supplements and dosage regimen.

In an embodiment, the collected user health data includes multiple user health parameters responsive to the nutraceutical polyphenol supplements and dosage regimen tracked simultaneously over time.

In another embodiment, the user health parameters are selected based on the type of risk being calculated for a user.

In another embodiment, the method further comprises calculating a risk of developing or suffering a medical condition within a set timeframe based on observed changes in the user's health condition resulting from the nutraceutical polyphenol supplements and dosage regimen.

In another embodiment, the risk calculation is made by an AI/ML inference module based on any or all of the user's health data as collected for the baseline and any subsequent measurements.

In another embodiment, the method further comprises collecting image data of a user's skin condition over a period of to assess skin texture and appearance in response to the nutraceutical polyphenol supplements and dosage regimen.

In another embodiment, any improvements to the user's skin condition are assessed by an AI/ML inference module based on comparing the collected image data of the user's skin condition against a model.

In another embodiment, the method further comprises tracking a group of users by anonymizing their data, and showing efficacy of the nutraceutical polyphenol supplements and dosage regimen for the group of users.

In another embodiment, the AI/ML inference module is adapted to learn from the data collected for a group of users, and project long-term health improvements for the group of users taking the nutraceutical polyphenol supplements and dosage regimen.

In another embodiment, the method further comprises recommending a change in the nutraceutical polyphenol supplements or the dosage regimen based on the long-term health of a group of users.

In another aspect, there is provided a system for assessing the efficacy of a nutraceutical polyphenol supplements and dosage regimen, the system having a processor, a memory, and storage, and adapted to: collect user health data from one or more biosensors, and from health assessment surveys executed on the computing device to establish a baseline for a user's health condition; collect data on the nutraceutical polyphenol supplements and dosage regimen a user is taking over time; and monitor the user's health condition at regular intervals using one or more biosensors and health assessment surveys, and comparing the user's updated health condition against the user's baseline to determine efficacy of the nutraceutical polyphenol supplements and dosage regimen.

In an embodiment, the collected user health data includes multiple user health parameters responsive to the nutraceutical polyphenol supplements and dosage regimen tracked simultaneously over time.

In another embodiment, the user health parameters are selected based on the type of risk being calculated for a user.

In another embodiment, the system is further adapted to calculate a risk of developing or suffering a medical condition within a set timeframe based on observed changes in the user's health condition resulting from the nutraceutical polyphenol supplements and dosage regimen.

In another embodiment, the risk calculation is made by an AI/ML inference module based on any or all of the user's health data as collected for the baseline and any subsequent measurements.

In another embodiment, the system is further adapted to collect image data of a user's skin condition over a period of time to assess skin texture and appearance in response to the nutraceutical polyphenol supplements and dosage regimen.

In another embodiment, any improvements to the user's skin condition are assessed by an AI/ML inference module based on comparing the collected image data of the user's skin condition against a model.

In another embodiment, the system is further adapted to track a group of users by anonymizing their data, and showing efficacy of the nutraceutical polyphenol supplements and dosage regimen for the group of users.

In another embodiment, the AI/ML inference module is adapted to learn from the data collected for a group of users, and project long-term health improvements for the group of users taking the nutraceutical polyphenol supplements and dosage regimen.

In another embodiment, the system is further adapted to recommend a change in the nutraceutical polyphenol supplements or the dosage regimen based on the long-term health of a group of users.

While various illustrative embodiments of the system, method, and apparatus have been described, it will be appreciated that various modifications and amendments may be made without departing from the scope of the invention.

1. A computer-implemented method of assessing the efficacy of a nutraceutical polyphenol supplements and dosage regimen, the method executable on a computing device having a processor, a memory, and storage, and comprising:
collecting user health data from one or more biosensors, and from health assessment surveys executed on the computing device to establish a baseline for a user's health condition;
collecting data on the nutraceutical polyphenol supplements and dosage regimen a user is taking over time; and
monitoring the user's health condition at regular intervals using one or more biosensors and health assessment surveys, and comparing the user's updated health condition against the user's baseline to determine efficacy of the nutraceutical polyphenol supplements and dosage regimen.

2. The computer-implemented method of claim 1, wherein the collected user health data includes multiple user health parameters responsive to the nutraceutical polyphenol supplements and dosage regimen tracked simultaneously over time.

3. The computer-implemented method of claim 2, wherein the user health parameters are selected based on the type of risk being calculated for a user.

4. The computer-implemented method of claim 1, further comprising calculating a risk of developing or suffering a medical condition within a set timeframe based on observed changes in the user's health condition resulting from the nutraceutical polyphenol supplements and dosage regimen.

5. The computer-implemented method of claim 4, wherein the risk calculation is made by an AI/ML inference module based on any or all of the user's health data as collected for the baseline and any subsequent measurements.

6. The computer-implemented method of claim 1, further comprising collecting image data of a user's skin condition over a period of to assess skin texture and appearance in response to the nutraceutical polyphenol supplements and dosage regimen.

7. The computer-implemented method of claim 6, wherein any improvements to the user's skin condition are assessed by an AI/ML inference module based on comparing the collected image data of the user's skin condition against a model.

8. The computer-implemented method of claim 1, further comprising tracking a group of users by anonymizing their data, and showing efficacy of the nutraceutical polyphenol supplements and dosage regimen for the group of users.

9. The computer-implemented method of claim 8, wherein the AI/ML inference module is adapted to learn from the data collected for a group of users, and project long-term health improvements for the group of users taking the nutraceutical polyphenol supplements and dosage regimen.

10. The computer-implemented method of claim 9, wherein the method further comprises recommending a change in the nutraceutical polyphenol supplements or the dosage regimen based on the long-term health of a group of users.

11. A system for assessing the efficacy of a nutraceutical polyphenol supplements and dosage regimen, the system having a processor, a memory, and storage, and adapted to:
collect user health data from one or more biosensors, and from health assessment surveys executed on the computing device to establish a baseline for a user's health condition;
collect data on the nutraceutical polyphenol supplements and dosage regimen a user is taking over time; and
monitor the user's health condition at regular intervals using one or more biosensors and health assessment surveys, and comparing the user's updated health condition against the user's baseline to determine efficacy of the nutraceutical polyphenol supplements and dosage regimen.

12. The system of claim 11, wherein the collected user health data includes multiple user health parameters responsive to the nutraceutical polyphenol supplements and dosage regimen tracked simultaneously over time.

13. The system of claim 12, wherein the user health parameters are selected based on the type of risk being calculated for a user.

14. The system of claim 11, wherein the system is further adapted to calculate a risk of developing or suffering a medical condition within a set timeframe based on observed changes in the user's health condition resulting from the nutraceutical polyphenol supplements and dosage regimen.

15. The system of claim 14, wherein the risk calculation is made by an AI/ML inference module based on any or all of the user's health data as collected for the baseline and any subsequent measurements.

16. The system of claim 11, wherein the system is further adapted to collect image data of a user's skin condition over a period of to assess skin texture and appearance in response to the nutraceutical polyphenol supplements and dosage regimen.

17. The system of claim 16, wherein any improvements to the user's skin condition are assessed by an AI/ML inference module based on comparing the collected image data of the user's skin condition against a model.

18. The system of claim 11, wherein the system is further adapted to track a group of users by anonymizing their data, and showing efficacy of the nutraceutical polyphenol supplements and dosage regimen for the group of users.

19. The system of claim 18, wherein the AI/ML inference module is adapted to learn from the data collected for a group of users, and project long-term health improvements for the group of users taking the nutraceutical polyphenol supplements and dosage regimen.

20. The system of claim 19, wherein the system is further adapted to recommend a change in the nutraceutical polyphenol supplements or the dosage regimen based on the long-term health of a group of users.
